(19) United States
(12) Patent Application Publication
(10) Pub. No.: US 2018/0013143 A1
(43) Pub. Date: Jan. 11, 2018

(54) SECONDARY BATTERY AND METHOD OF MANUFACTURING THE SAME, BATTERY PACK, ELECTRIC VEHICLE, ELECTRIC POWER STORAGE SYSTEM, ELECTRIC POWER TOOL, AND ELECTRONIC APPARATUS

(71) Applicants: Sony Corporation, Tokyo (JP); Hydro-Quebec, Montreal (CA)

(72) Inventors: Yuichiro ASAKAWA, Saitama (JP); Jean-Christophe DAIGLE, Quebec (CA); Karim ZAGHIB, Quebec (CA); Shinichi UESAKA, Quebec (CA)

(21) Appl. No.: 15/631,881

(22) Filed: Jun. 23, 2017

Related U.S. Application Data
(60) Provisional application No. 62/358,940, filed on Jul. 6, 2016.

(51) Int. Cl. H01M 4/485; C01G 33/00; C01G 25/00; C01G 31/00; H01M 10/056; C07D 317/40

(52) U.S. Cl. CPC H01M 4/485; H01M 10/056; C07D 317/40; C01G 25/006; C01G 31/006; C01G 33/006

(57) ABSTRACT

There is provided a secondary battery including a cathode, an anode including an anode active material layer and a coating film, and an electrolytic solution. The anode active material layer includes a titanium-containing compound, and a surface of the anode active material layer is coated with the coating film. The electrolytic solution includes one or more of unsaturated cyclic carbonate esters. Porosity of a portion of the anode active material layer measured with use of a mercury intrusion technique is within a range from 30% to 50% both inclusive. The portion of the anode active material layer is cut together with a portion of the coating film from a surface of the coating film to a depth of 10 μm.

### CROSS-REFERENCE TO RELATED APPLICATIONS

The present application claims priority from U.S. Provisional Application Ser. No. 62/358,940 filed on Jul. 6, 2016, the content of which is hereby incorporated by reference into this application.

### TECHNICAL FIELD OF THE INVENTION

The present technology relates to a secondary battery that uses an anode including a titanium-containing compound and a method of manufacturing the same, and to a battery pack, an electric vehicle, an electric power storage system, an electric power tool, and an electronic apparatus each of which uses the secondary battery.

### BACKGROUND OF THE INVENTION

Various electronic apparatuses such as mobile phones have been widely used, and it has been demanded to further reduce size and weight of the electronic apparatuses and to achieve their longer lives. Accordingly, small and light-weight secondary batteries that have ability to achieve high energy density have been developed as power sources for the electronic apparatuses.

Applications of the secondary batteries are not limited to the electronic apparatuses described above, and it has been also considered to apply the secondary batteries to various other applications. Examples of such other applications may include: a battery pack attachably and detachably mounted on, for example, an electronic apparatus; an electric vehicle such as an electric automobile; an electric power storage system such as a home electric power server; and an electric power tool such as an electric drill.

The secondary battery includes a cathode, an anode, and electrolytic solution. The configuration of the secondary battery exerts a large influence on battery characteristics. Accordingly, various studies have been conducted on the configuration of the secondary battery.

More specifically, in order to improve characteristics such as cycle characteristics, a lithium-titanium composite oxide (Li_{4/3}Ti_{5/3}O₄) is used as an anode active material, and an unsaturated cyclic carbonate ester (vinylene carbonate) is used as an additive of an electrolytic solution (refer to High Temperature Life Performance for Lithium-ion Battery Using Lithium Titanium Oxide Negative Electrode with Electrochemically Formed Surface Film Comprising Organic-Inorganic Binary Constituents, GS Yuasa Technical Report, June 2009, Vol. 6, No. 1). In this case, in order to examine characteristics such as cycle characteristics, an ambient temperature is set to 80° C.

### SUMMARY OF THE INVENTION

Specific proposals have been made in order to improve battery characteristics of the secondary battery; however, the battery characteristics of the secondary battery are not sufficient yet. For this reason, there is still room for improvement.

It is therefore desirable to provide a secondary battery that makes it possible to achieve superior battery characteristics and a method of manufacturing the same, and a battery pack, an electric vehicle, an electric power storage system, an electric power tool, and an electronic apparatus.

According to an embodiment of the present technology, there is provided a secondary battery including: a cathode; an anode including an anode active material layer and a coating film, the anode active material layer including a titanium-containing compound, and a surface of the anode active material layer being coated with the coating film; and an electrolytic solution including one or more of respective unsaturated cyclic carbonate esters represented by the following formulas (11) to (13). Porosity of a portion of the anode active material layer measured with use of a mercury intrusion technique is within a range from 30% to 50% both inclusive, and the portion of the anode active material layer is cut together with a portion of the coating film from a surface of the coating film to a depth of 10 μm,

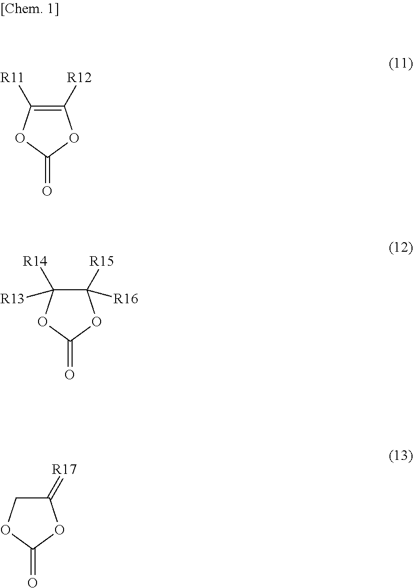

where each of R11 and R12 is one of a hydrogen group and an alkyl group, each of R13 to R16 is one of a hydrogen group, an alkyl group, a vinyl group, and an allyl group, one or more of R13 to R16 are one of the vinyl group and the allyl group, R17 is a group represented by >CR171R172, and each of R171 and R172 is one of a hydrogen group and an alkyl group.

According to an embodiment of the present technology, there is provided a method of manufacturing a secondary battery including: fabricating a secondary battery including a cathode, an anode, and an electrolytic solution, the anode including an anode active material layer that includes a titanium-containing compound, and the electrolytic solution including one or more of respective unsaturated cyclic carbonate esters represented by the foregoing formulas (11) to (13); charging and discharging the secondary battery to form a coating film, a surface of the anode active material layer being coated with the coating film; and performing heat treatment on the secondary battery, in which the coating film is formed on the surface of the anode active material layer, at a treatment temperature ranging from 45° C. to 60° C. both inclusive for treatment time ranging from 12 hours to 100 hours both inclusive in a state of charge ranging from 25% to 75% both inclusive.

According to respective embodiments of the present technology, there are provided a battery pack, an electric vehicle, an electric power storage system, an electric power tool, and an electronic apparatus each including a secondary battery, and the secondary battery has a configuration similar to that of the secondary battery according to the foregoing embodiment of the present technology.

Herein, in order to cut the portion of the anode active material layer together with the portion of the coating film, for example, a surface and interfacial cutting Analysis system (SAICAS) may be used.

Moreover, the porosity of the portion of the anode active material layer may be measured with use of, for example, a mercury porosimeter using the mercury intrusion technique. In this case, surface tension of mercury is equal to 485 mN/m, a contact angle of mercury is equal to 130°, and a relationship between a pore diameter of a pore and pressure is approximate to 180/pressure=the pore diameter. The mercury porosimeter may be, for example, a mercury porosimeter (AutoPore 9500 series) available from Micromeritics Instrument Corp., located in U.S.A.

According to the secondary battery of the embodiment of the present technology, the foregoing porosity of the portion of the anode active material layer is within a range from 30% to 50% both inclusive, which makes it possible to achieve superior battery characteristics. Moreover, in each of the battery pack, the electric vehicle, the electric power storage system, the electric power tool, and the electronic apparatus of the respective embodiments of the present technology, similar effects are achievable.

Moreover, according to the method of manufacturing the secondary battery of the embodiment of the present technology, the secondary battery including the anode that includes the anode active material layer including the titanium-containing compound, and the electrolytic solution including the one or more of the unsaturated cyclic carbonate esters is fabricated, and the secondary battery is charged and discharged to form the coating film, and thereafter, the heat treatment is performed on the secondary battery under the foregoing conditions, which makes it possible to easily and stably manufacture the secondary battery in which the foregoing porosity of the portion of the anode active material layer is within a range from 30% to 50% both inclusive.

Note that effects described here are non-limiting. Effects achieved by the present technology may be one or more of effects described in the present technology.

It is to be understood that both the foregoing general description and the following detailed description are exemplary, and are provided to provide further explanation of the technology as claimed.

### BRIEF DESCRIPTION OF THE DRAWINGS

The accompanying drawings are included to provide a further understanding of the technology, and are incorporated in and constitute a part of this specification. The drawings illustrate embodiments and, together with the specification, serve to explain the principles of the technology.

FIG. 1 is a cross-sectional view of a configuration of a secondary battery (cylindrical type) according to an embodiment of the present technology.

FIG. 2 is a cross-sectional view of part of a spirally wound electrode body illustrated in FIG. 1.

FIG. 3 is a cross-sectional view for description of a procedure of cutting an anode.

FIG. 4 is a perspective view of a configuration of another secondary battery (laminated film type) according to the embodiment of the present technology

FIG. 5 is a cross-sectional view taken along a line V-V of a spirally wound electrode body illustrated in FIG. 4.

FIG. 6 is an enlarged cross-sectional view of part of a configuration of the spirally wound electrode body illustrated in FIG. 5.

FIG. 7 is a perspective view of a configuration of an application example (a battery pack: single battery) of the secondary battery.

FIG. 8 is a block diagram illustrating a configuration of the battery pack illustrated in FIG. 7.

FIG. 9 is a block diagram illustrating a configuration of an application example (a battery pack: assembled battery) of the secondary battery.

FIG. 10 is a block diagram illustrating a configuration of an application example (an electric vehicle) of the secondary battery.

FIG. 11 is a block diagram illustrating a configuration of an application example (an electric power storage system) of the secondary battery.

FIG. 12 is a block diagram illustrating a configuration of an application example (an electric power tool) of the secondary battery.

FIG. 13 is a cross-sectional view of a configuration of a test-use secondary battery (coin type).

### DETAILED DESCRIPTION

In the following, some embodiments of the present technology are described in detail with reference to drawings. It is to be noted that description is given in the following order.

1. Secondary Battery (Cylindrical Type)
1-1. Configuration
1-2. Physical Properties of Anode
1-3. Operation
1-4. Manufacturing Method
1-5. Action and Effects

2. Secondary Battery (Laminated Film Type)
2-1. Configuration
2-2. Operation
2-3. Manufacturing Method
2-4. Action and Effects

3. Applications of Secondary Battery
3-1. Battery Pack (Single Battery)
3-2. Battery Pack (Assembled Battery)
3-3. Electric Vehicle
3-4. Electric Power Storage System
3-5. Electric Power Tool

### <1. Secondary Battery (Cylindrical Type)>

First, description is given of a secondary battery according to an embodiment of the present technology.

The secondary battery described here may be, for example, a lithium-ion secondary battery in which a battery capacity (a capacity of an anode) is obtained with use of a lithium insertion phenomenon and a lithium extraction phenomenon.

### <1-1. Configuration>

First, description is given of a configuration of the secondary battery.

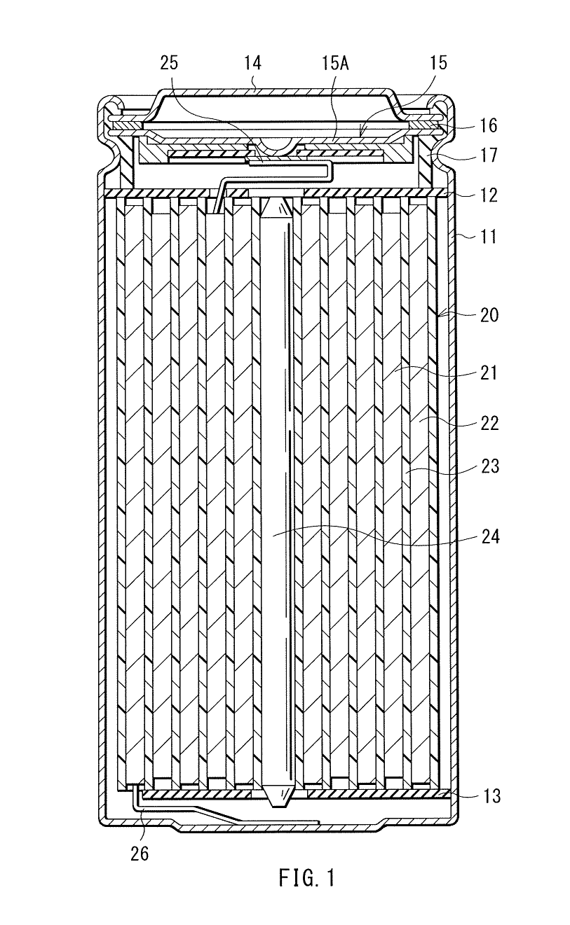

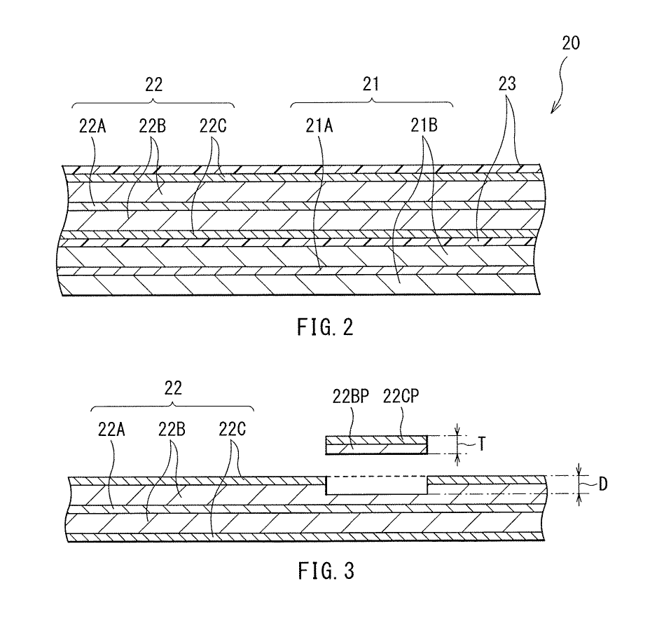

FIG. 1 illustrates a cross-sectional configuration of a secondary battery. FIG. 2 is an enlarged view of part of a cross-sectional configuration of a spirally wound electrode body 20 illustrated in FIG. 1. As can be seen from FIG. 1, the secondary battery may be, for example, a so-called cylindrical type secondary battery.

### [Whole Configuration]

Specifically, the secondary battery may contain, for example, a pair of insulating plates 12 and 13 and the spirally wound electrode body 20 as a battery element inside a battery can 11 having a substantially hollow cylindrical shape, as illustrated in FIG. 1. The spirally wound electrode body 20 may be formed as follows. For example, a cathode 21 and the anode 22 may be stacked with a separator 23 in between, and the cathode 21, the anode 22, and the separator 23 may be spirally wound to form the spirally wound electrode body 20. The spirally wound electrode body 20 may be impregnated with, for example, an electrolytic solution that is a liquid electrolyte.

The battery can 11 may have, for example, a hollow structure in which one end of the battery can 11 is closed and the other end of the battery can 11 is open. The battery can 11 may include, for example, one or more of iron, aluminum, and an alloy thereof. A surface of the battery can 11 may be plated with, for example, a metal material such as nickel. Note that the pair of insulating plates 12 and 13 may be so disposed as to sandwich the spirally wound electrode body 20 in between and extend perpendicularly to a spirally wound periphery surface of the spirally wound electrode body 20.

At the open end of the battery can 11, a battery cover 14, a safety valve mechanism 15, and a positive temperature coefficient device (PTC device) 16 may be swaged with a gasket 17, by which the battery can 11 is hermetically sealed. A formation material of the battery cover 14 may be similar to, for example, a formation material of the battery can 11. Each of the safety valve mechanism 15 and the PTC device 16 may be provided on the inner side of the battery cover 14, and the safety valve mechanism 15 may be electrically coupled to the battery cover 14 via the PTC device 16. In the safety valve mechanism 15, when an internal pressure of the battery can 11 reached a certain level or higher as a result of, for example, internal short circuit or heating from outside, a disk plate 15A inverts. This cuts electric connection between the battery cover 14 and the spirally wound electrode body 20. In order to prevent abnormal heat generation resulting from a large current, resistance of the PTC device 16 increases as a temperature rises. The gasket 17 may include, for example, an insulating material. A surface of the gasket 17 may be coated with, for example, asphalt.

For example, A center pin 24 may be inserted in a space provided at a center of the spirally wound electrode body 20. However, the center pin 24 may be omitted.

A cathode lead 25 may be attached to the cathode 21. The cathode lead 25 may include, for example, a conductive material such as aluminum. For example, the cathode lead 25 may be attached to the safety valve mechanism 15, which may be thereby electrically coupled to the battery cover 14.

An anode lead 26 may be attached to the anode 22. The anode lead 26 may include, for example, a conductive material such as nickel. For example, the anode lead 26 may be attached to the battery can 11, which may be thereby electrically coupled to the battery can 11.

### [Cathode]

The cathode 21 may include, for example, a cathode current collector 21A and two cathode active material layers 21B provided on both surfaces of the cathode current collector 21A. Alternatively, only one cathode active material layer 21B may be provided on a single surface of the cathode current collector 21A.

### (Cathode Current Collector)

The cathode current collector 21A may include, for example, one or more of conductive materials. The kind of the conductive materials is not particularly limited; however, non-limiting examples of the conductive materials may include metal materials such as aluminum, nickel, and stainless steel. The cathode current collector 21A may be configured of a single layer or may be configured of multiple layers.

### (Cathode Active Material Layer)

The cathode active material layer 21B may contain, as a cathode active material, one or more of materials (cathode materials) that have ability to insert and extract lithium. It is to be noted that the cathode active material layer 21B may further include one or more of other materials such as a cathode binder and a cathode conductor.

### (Cathode Material: Lithium-containing Compound)

The cathode material may include, for example, one or more of lithium-containing compounds, which makes it possible to achieve high energy density. The kind of the lithium-containing compounds is not particularly limited; however, non-limiting examples of the lithium-containing compounds may include a lithium-containing composite oxide and a lithium-containing phosphate compound.

The “lithium-containing composite oxide” is a generic name of an oxide that includes lithium (Li) and one or more other elements as constituent elements. The lithium-containing composite oxide may have, for example, one of crystal structures such as a layered rock-salt crystal structure and a spinel crystal structure.

The “lithium-containing phosphate compound” is a generic name of a phosphate compound that includes lithium and one or more other elements as constituent elements. The lithium-containing phosphate compound may have, for example, a crystal structure such as an olivine crystal structure.

It is to be noted that the “other elements” are elements other than lithium. The kind of the other elements is not particularly limited; however, non-limiting examples of the other elements may include elements that belong to Groups 2 to 15 in the long form of the periodic table of the elements. Specific but non-limiting examples of the other elements may include nickel (Ni), cobalt (Co), manganese (Mn), and iron (Fe), which make it possible to obtain a high voltage.

Non-limiting examples of the lithium-containing composite oxide having the layered rock-salt crystal structure may include compounds represented by the following formulas (21) to (23).

LiₐMn_{(1-b-c)}Ni_{b}M11_{c}O_{(2-d)}Fₑ (21)

where M11 is one or more of cobalt (Co), magnesium (Mg), aluminum (Al), boron (B), titanium (Ti), vanadium (V), chromium (Cr), iron (Fe), copper (Cu), zinc (Zn), zirconium (Zr), molybdenum (Mo), tin (Sn), calcium (Ca), strontium (Sr), and tungsten (W), “a” to “e” satisfy 0.8≦a≦1.2, 0<b<0.5, 0≦c≦0.5, (b+c)<1, −0.1≦d≦0.2,0 and 0≦e≦0.1, it is to be noted that the composition of lithium varies depending on charge and discharge states, and “a” is a value in a completely-discharged state.

LiₐNi_{(1-b)}M12_{b}O_{(2-c)}F_{d} (22)

where M12 is one or more of cobalt (Co), manganese (Mn), magnesium (Mg), aluminum (Al), boron (B), titanium (Ti), vanadium (V), chromium (Cr), iron (Fe), copper (Cu), zinc (Zn), molybdenum (Mo), tin (Sn), calcium (Ca), strontium (Sr), and tungsten (W), “a” to “d” satisfy 0.8≦a≦1.2, 0.0005≦b≦0.5, −0.1≦c≦0.2, and 0≦d≦0.1, it is to be noted that the composition of lithium varies depending on charge and discharge states, and “a” is a value in a completely-discharged state.

LiₐCo_{(1-b)}M13_{b}O_{(2-c)}F_{d} (23)

where M13 is one or more of nickel (Ni), manganese (Mn), magnesium (Mg), aluminum (Al), boron (B), titanium (Ti), vanadium (V), chromium (Cr), iron (Fe), copper (Cu), zinc (Zn), molybdenum (Mo), tin (Sn), calcium (Ca), strontium (Sr), and tungsten (W), “a” to “d” satisfy 0.8≦a≦1.2, 0≦b≦0.5, −0.1≦c≦0.2, and 0≦d≦0.1, it is to be noted that the composition of lithium varies depending on charge and discharge states, and “a” is a value in a completely-discharged state.

Specific but non-limiting examples of the lithium-containing composite oxide having the layered rock-salt crystal structure may include LiNiO₂, LiCoO₂, LiCo_{0.980}Al_{0.01}Mg_{0.01}O₂, LiNi_{0.5}Co_{0.2}Mn_{0.3}O₂, LiNi_{0.8}Co_{0.15}Al_{0.05}O₂, LiNi_{.033}Co_{0.33}Mn_{0.33}O₂, Li_{1.2}Mn_{0.52}Co_{0.175}Ni_{0.1}O₂ , and Li_{1.15}(Mn_{0.65}Ni_{0.22}Co_{0.13})O₂.

It is to be noted that in a case where the lithium-containing composite oxide having the layered rock-salt crystal structure includes nickel, cobalt, manganese, and aluminum as constituent elements, an atomic ratio of nickel may be preferably 50 at % or more, which makes it possible to achieve high energy density.

Non-limiting examples of the lithium-containing composite oxide having the spinel crystal structure may include a compound represented by the following formula (24).

LiₐMn_{(2-b)}M14_{b}O_{c}F_{d} (24)

where M14 is one or more of cobalt (Co), nickel (Ni), magnesium (Mg), aluminum (Al), boron (B), titanium (Ti), vanadium (V), chromium (Cr), iron (Fe), copper (Cu), zinc (Zn), molybdenum (Mo), tin (Sn), calcium (Ca), strontium (Sr), and tungsten (W), “a” to “d” satisfy 0.9≦a≦1.1, 0≦b≦0.6, 3.7≦c≦4.1, and 0≦d≦0.1, it is to be noted that the composition of lithium varies depending on charge and discharge states, and “a” is a value in a completely-discharged state.

Specific but non-limiting examples of the lithium-containing composite oxide having the spinel crystal structure may include LiMn₂O₄.

Non-limiting examples of the lithium-containing phosphate compound having the olivine crystal structure may include a compound represented by the following formula (25).

LiₐM15PO₄ (25)

where M15 is one or more of cobalt (Co), manganese (Mn), iron (Fe), nickel (Ni), magnesium (Mg), aluminum (Al), boron (B), titanium (Ti), vanadium (V), niobium (Nb), copper (Cu), zinc (Zn), molybdenum (Mo), calcium (Ca), strontium (Sr), tungsten (W), and zirconium (Zr), “a” satisfies 0.9≦a≦1.1, it is to be noted that the composition of lithium varies depending on charge and discharge states, and “a” is a value in a completely-discharged state.

Specific but non-limiting examples of the lithium-containing phosphate compound having the olivine crystal structure may include LiFePO₄, LiMnPO₄, LiFe_{0.5}Mn_{0.5}PO₄, and LiFe_{0.3}Mn_{0.7}PO₄.

It is to be noted that the lithium-containing composite oxide may be, for example, a compound represented by the following formula (26).

(Li₂MnO₃)ₓ(LiMnO₂)₁₋ₓ (26)

where “x” satisfies it is to be noted that the composition of lithium varies depending on charge and discharge states, and “x” is a value in a completely-discharged state.

### (Other Cathode Materials)

It is to be noted that the cathode material may include one or more of other cathode materials together with the foregoing titanium-containing compound. The kind of the other cathode materials is not particularly limited; however, non-limiting examples of the other cathode materials may include an oxide, a disulfide, a chalcogenide, and a conductive polymer.

Non-limiting examples of the oxide may include titanium oxide, vanadium oxide, and manganese dioxide. Non-limiting examples of the disulfide may include titanium disulfide and molybdenum sulfide. Non-limiting examples of the chalcogenide may include niobium selenide. Non-limiting examples of the conductive polymer may include sulfur, polyaniline, and polythiophene.

### (Cathode Binder)

The cathode binder may include, for example, one or more of synthetic rubbers and polymer materials. Non-limiting examples of the synthetic rubbers may include a styrene-butadiene-based rubber, a fluorine-based rubber, and ethylene propylene diene. Non-limiting examples of the polymer materials may include polyvinylidene fluoride and polyimide.

### (Cathode Conductor)

The cathode conductor may include, for example, one or more of carbon materials. Non-limiting examples of the carbon materials may include graphite, carbon black, acetylene black, and Ketjen black. Alternatively, the cathode conductor may be any other material such as a metal material and a conductive polymer, as long as the cathode conductor is a material having conductivity.

### [Anode]

The anode 22 may include, for example, an anode current collector 22A, two anode active material layers 22B provided on both surfaces of the anode current collector 22A, and two coating films 22C with which surfaces of the two anode active material layers 22B are coated. Alternatively, only one anode active material layer 22B may be provided on a single surface of the anode current collector 22A. Moreover, in a case where the two anode active material layers 22B are provided on both surface of the anode current collector 22A, only one coating film 22C may be provided on the surface of one of the two anode active material layers 22B.

### (Anode Current Collector)

The anode current collector 22A may include, for example, one or more of conductive materials. The kind of the conductive material is not particularly limited, but may be, for example, a metal material such as copper, aluminum, nickel, and stainless steel. The anode current collector 22A may be configured of a single layer or may be configured of multiple layers.

A surface of the anode current collector 22A may be preferably roughened. This makes it possible to improve adhesibility of the anode active material layers 22B with respect to the anode current collector 22A by a so-called anchor effect. In this case, it may be only necessary to roughen the surface of the anode current collector 22A at least in a region facing each of the anode active material layers 22B. Non-limiting examples of a roughening method may include a method of forming fine particles with use of electrolytic treatment. Through the electrolytic treatment, fine particles are formed on the surface of the anode current collector 22A in an electrolytic bath by an electrolytic method to make the surface of the anode current collector 22A rough. A copper foil fabricated by the electrolytic method is generally called “electrolytic copper foil”.

### (Anode Active Material Layer)

The anode active material layers 22B may include, as an anode active material, one or more of materials (anode materials) that have ability to insert and extract lithium. It is to be noted that the anode active material layers 22B may further include one or more of other materials such as an anode binder and an anode conductor.

In order to prevent lithium from being unintentionally precipitated on the anode 22 in the middle of charge, chargeable capacity of the anode material may be preferably larger than discharge capacity of the cathode 21. In other words, electrochemical equivalent of the anode material that has ability to insert and extract lithium may be preferably larger than electrochemical equivalent of the cathode 21.

The thickness of the anode active material layer 22B is not particularly limited, but may be within a range from 30 μm to 100 μm both inclusive.

### (Anode Material: Titanium-containing Compound)

The anode material includes one or more of titanium-containing compounds. The kind of the titanium-containing compounds is not particularly limited; however, non-limiting examples of the titanium-containing compounds may include a titanium oxide, a lithium-titanium composite oxide, and a hydrogen-titanium compound. Since the titanium-containing compounds are electrochemically stable (have low reactivity), as compared with carbon materials, etc. to be described later, the titanium-containing compounds suppress decomposition reaction of the electrolytic solution resulting from reactivity of the anode 22.

The “titanium oxide” is a generic name of a compound of titanium (Ti) and oxygen (O).

The “lithium-titanium composite oxide” is a generic name of an oxide including titanium and one or more of other elements as constituent elements. Details of the other elements may be as described above, for example.

The “hydrogen-titanium compound” is a generic name of a compound including hydrogen (H) and titanium as constituent elements. Note that the hydrogen-titanium compound described here is excluded from the foregoing lithium-titanium composite oxide.

More specifically, the titanium oxide may include, for example, a compound represented by the following formula (1). More specifically, non-limiting examples of the titanium oxide may include a bronze type titanium oxide.

TiO_{w} (1)

where w satisfies 1.85≦w≦2.15.

Specific but non-limiting examples of the titanium oxide may include anatase type, rutile type, and brookite type titanium oxides (TiO₂).

Note that the titanium oxide may be a composite oxide including, together with titanium, one or more of elements such as phosphorus (P), vanadium (V), tin (Sn), copper (Cu), nickel (Ni), iron (Fe), and cobalt (Co). Specific but non-limiting examples of the composite oxide may include TiO₂-P₂O₅, TiO₂-V₂O₅, TiO₂-P₂O₅-SnO₂, and TiO₂-P₂O₅-MeO, where Me may be, for example, one or more of elements such as copper, nickel, iron, and cobalt.

A potential at which lithium is inserted in and extracted from these titanium oxides may be, for example, within a range from 1 V to 2 V both inclusive (vs Li/Li⁺).

The lithium-titanium composite oxide may include, for example, one or more of respective compounds represented by the following formulas (2) to (4). More specifically, non-limiting examples of the lithium-titanium composite oxide may include a ramsdellite type lithium titanate. M1 in the formula (2) is a metal element that possibly becomes a divalent ion. M2 in the formula (3) is a metal element that possibly becomes a trivalent ion. M3 in the formula (4) is a metal element that possibly becomes a tetravalent ion.

Li[LiₓM1_{(1-3x/2}Ti_{3+x)/2}]O₄ (2)

where M1 is one or more of magnesium (Mg), calcium (Ca), copper (Cu), zinc (Zn), and strontium (Sr), and “x” satisfies O)(1/3.

Li[Li_{y}M2_{1-3y}Ti_{1+2y}]O₄ (3)

where M2 is one or more of aluminum (Al), scandium (Sc), chromium (Cr), manganese (Mn), iron (Fe), germanium (Ga), and yttrium (Y), and “y” satisfies 0≦y≦1/3.

Li[Li_{1/3}M3_{z}Ti_{(5/3)-z}]O₄ (4)

where M3 is one or more of vanadium (V), zirconium (Zr), and niobium (Nb), and “z” satisfies 0≦z≦2/3.

The crystal structure of the lithium-titanium composite oxide is not particularly limited; however, in particular, the spinel type crystal structure may be preferable. The spinel type crystal structure is resistant to change during charge and discharge, which makes it possible to achieve stable battery characteristics.

Specific but non-limiting examples of the compound represented by the formula (2) may include Li_{3.75}Ti_{4.875}Mg_{0.375}O₁₂. Specific but non-limiting examples of the compound represented by the formula (3) may include LiCrTiO₄. Specific but non-limiting examples of the compound represented by the formula (4) may include Li₄Ti₅O₁₂ and Li₄Ti_{4.95}Nb_{0.05}O₁₂.

Specific but non-limiting examples of the hydrogen-titanium compound may include H₂Ti₃O₇(3TiO₂.1H₂O), H₆Ti₁₂O₂₇(3Ti O₂.0.75H₂O), H₂Ti₆O₁₃(3TiO₂.0.5H₂O), H₂Ti₇O₁₅(3TiO₂.0.43H₂O), and H₂Ti₁₂O₂₅(3TiO₂.0.2 5H₂O).

It goes without saying that two or more of the respective compounds represented by the formulas (2) to (4) may be used in combination. Moreover, the titanium oxide and the lithium-titanium composite oxide may be used in combination.

### (Other Anode Materials)

It is to be noted that the anode material may include one or more of other anode materials together with the foregoing lithium-titanium composite oxide. The kind of the other anode materials is not particularly limited; however, non-liming examples of the other anode materials may include a carbon material and a metal-based material.

The “carbon material” is a generic name of a material including carbon as a constituent element. The carbon material causes an extremely-small change in a crystal structure thereof during insertion and extraction of lithium, which stably achieves high energy density. Further, the carbon material also serves as the anode conductor, which improves conductivity of the anode active material layer 22B.

Non-limiting examples of the carbon material may include graphitizable carbon, nongraphitizable carbon, and graphite. A spacing of (002) plane in the nongraphitizable carbon may be preferably 0.37 nm or larger, and a spacing of (002) plane in the graphite may be preferably 0.34 nm or smaller. More specific examples of he carbon material may include pyrolytic carbons, cokes, glassy carbon fibers, an organic polymer compound fired body, activated carbon, and carbon blacks. Non-limiting examples of the cokes may include pitch coke, needle coke, and petroleum coke. The organic polymer compound fired body is a polymer compound fired (carbonized) at an appropriate temperature. Non-limiting examples of the polymer compound may include phenol resin and furan resin. Other than the materials mentioned above, the carbon material may be low crystalline carbon that is subjected to heat treatment at a temperature of about 1000° C. or lower, or may be amorphous carbon. It is to be noted that a shape of the carbon material may be one or more of a fibrous shape, a spherical shape, a granular shape, and a scale-like shape.

The “metal-based material” is a generic name of a material including one or more of metal elements and metalloid elements as constituent elements, and the metal-based material achieves high energy density. However, the foregoing lithium-titanium composite oxide is excluded from the metal-based material described here.

The metal-based material may be any of a simple substance, an alloy, or a compound, may be two or more thereof, or may have one or more phases thereof at least in part. It is to be noted that the “alloy” also encompasses a material that includes one or more metal elements and one or more metalloid elements, in addition to a material that is configured of two or more metal elements. Further, the alloy may include one or more of nonmetallic elements. Non-limiting examples of a structure of the metal-based material may include a solid solution, a eutectic crystal (a eutectic mixture), an intermetallic compound, and a structure in which two or more thereof coexist.

The metal elements and the metalloid elements may be, for example, one or more of metal elements and metalloid elements that are able to form an alloy with lithium. Specific but non-limiting examples thereof may include magnesium (Mg), boron (B), aluminum (Al), gallium (Ga), indium (In), silicon (Si), germanium (Ge), tin (Sn), lead (Pb), bismuth (Bi), cadmium (Cd), silver (Ag), zinc, hafnium (Hf), zirconium, yttrium (Y), palladium (Pd), and platinum (Pt).

In particular, silicon, tin, or both may be preferable. Silicon and tin have superior ability to insert and extract lithium, and achieve remarkably high energy density accordingly.

A material that includes silicon, tin, or both as constituent elements may be any of a simple substance, an alloy, and a compound of silicon, may be any of a simple substance, an alloy, and a compound of tin, may be two or more thereof, or may be a material that has one or more phases thereof at least in part. The simple substance described here merely refers to a simple substance in a general sense (in which a small amount of impurity may be contained), and does not necessarily refer to a simple substance having a purity of 100%.

The alloy of silicon may include, for example, one or more of elements such as tin, nickel, copper, iron, cobalt, manganese, zinc, indium, silver, titanium, germanium, bismuth, antimony, and chromium, as constituent elements other than silicon. The compound of silicon may include, for example, one or more of elements such as carbon and oxygen, as constituent elements other than silicon. It is to be noted that the compound of silicon may include, for example, one or more of the elements described related to the alloy of silicon, as constituent elements other than silicon.

Specific but non-limiting examples of the alloy of silicon and the compound of silicon may include SiB₄, SiB₆, Mg₂Si, Ni₂Si, TiSi₂, MoSi₂, CoSi₂, NiSi₂, CaSi₂, CrSi₂, Cu₅Si, FeSi₂, MnSi₂, NbSi₂, TaSi₂, VSi₂, WSi₂, ZnSi₂, SiC, Si₃N₄, Si₂N₂O, SiOᵥ (0<v≦2), and LiSiO. It is to be noted that “v” in SiOᵥ may be, for example, within a range of 0.2<v<1.4.

The alloy of tin may include, for example, one or more of elements such as silicon, nickel, copper, iron, cobalt, manganese, zinc, indium, silver, titanium, germanium, bismuth, antimony, and chromium, as constituent elements other than tin. The compound of tin may include, for example, one or more of elements such as carbon and oxygen, as constituent elements other than tin. It is to be noted that the compound of tin may include, for example, one or more of the elements described related to the alloy of tin, as constituent elements other than tin.

Specific but non-limiting examples of the alloy of tin and the compound of tin may include SnO_{w} (0<w≦2), SnSiO₃, LiSnO, and Mg₂Sn.

In particular, the material that includes tin as a constituent element may be preferably, for example, a material (tin-containing material) that includes, together with tin as a first constituent element, a second constituent element and a third constituent element. The second constituent element may include, for example, one or more of elements such as cobalt, iron, magnesium, titanium, vanadium, chromium, manganese, nickel, copper, zinc, gallium, zirconium, niobium, molybdenum, silver, indium, cesium (Ce), hafnium (Hf), tantalum, tungsten, bismuth, and silicon. The third constituent element may include, for example, one or more of elements such as boron, carbon, aluminum, and phosphorus. The tin-containing material including the second constituent element and the third constituent element makes it possible to achieve, for example, high battery capacity and superior cycle characteristics.

In particular, the tin-containing material may be preferably a material (a tin-cobalt-carbon-containing material) that includes tin, cobalt, and carbon as constituent elements. In the tin-cobalt-carbon-containing material, for example, a content of carbon may be from 9.9 mass % to 29.7 mass % both inclusive, and a ratio of contents of tin and cobalt (Co/(Sn+Co)) may be from 20 mass % to 70 mass % both inclusive. This makes it possible to achieve high energy density.

The tin-cobalt-carbon-containing material may have a phase that includes tin, cobalt, and carbon. Such a phase may be preferably low crystalline or amorphous. This phase is a reaction phase that is able to react with lithium. Hence, existence of the reaction phase results in achievement of superior characteristics. A half width (a diffraction angle 2θ) of a diffraction peak obtained by X-ray diffraction of this reaction phase may be preferably 1° or larger in a case where a CuKα ray is used as a specific X-ray, and an insertion rate is 1° /min. This makes it possible to insert and extract lithium more smoothly, and to decrease reactivity with the electrolytic solution. It is to be noted that, in some cases, the tin-cobalt-carbon-containing material may include a phase that includes simple substances of the respective constituent elements or part thereof in addition to the low-crystalline phase or the amorphous phase.

Comparison between X-ray diffraction charts before and after an electrochemical reaction with lithium makes it possible to easily determine whether the diffraction peak obtained by the X-ray diffraction corresponds to the reaction phase that is able to react with lithium. For example, if a position of the diffraction peak after the electrochemical reaction with lithium is changed from the position of the diffraction peak before the electrochemical reaction with lithium, the obtained diffraction peak corresponds to the reaction phase that is able to react with lithium. In this case, for example, the diffraction peak of the low-crystalline reaction phase or the amorphous reaction phase may be seen within a range of 2θ that is from 20° to 50° both inclusive. Such a reaction phase may include, for example, the respective constituent elements mentioned above, and it may be considered that such a reaction phase has become low crystalline or amorphous mainly because of existence of carbon.

In the tin-cobalt-carbon-containing material, part or all of carbon that is the constituent element thereof may be preferably bound to one or both of a metal element and a metalloid element that are other constituent elements thereof. Binding part or all of carbon suppresses cohesion or crystallization of, for example, tin. It is possible to confirm a binding state of the elements, for example, by X-ray photoelectron spectroscopy (XPS). In a commercially-available apparatus, for example, an Al-Kα ray or a Mg-Kα ray may be used as a soft X-ray. In a case where part or all of carbon is bound to one or both of the metal element and the metalloid element, a peak of a synthetic wave of 1s orbit of carbon (C1s) appears in a region lower than 284.5 eV. It is to be noted that energy calibration is so made that a peak of 4f orbit of a gold atom (Au4f) is obtained at 84.0 eV. In this case, in general, surface contamination carbon exists on the material surface. Hence, a peak of C1s of the surface contamination carbon is regarded to be at 284.8 eV, and this peak is used as energy standard. In XPS measurement, a waveform of the peak of C1s is obtained as a form that includes the peak of the surface contamination carbon and the peak of the carbon in the tin-cobalt-carbon-containing material. The two peaks may be therefore separated from each other, for example, by analysis with use of commercially-available software. In the analysis of the waveform, a position of the main peak that exists on the lowest bound energy side is regarded as the energy standard (284.8 eV).

The tin-cobalt-carbon-containing material is not limited to a material that includes only tin, cobalt, and carbon as constituent elements. The tin-cobalt-carbon-containing material may further include one or more of, for example, silicon, iron, nickel, chromium, indium, niobium, germanium, titanium, molybdenum, aluminum, phosphorus, gallium, and bismuth, as constituent elements, in addition to tin, cobalt, and carbon.

Other than the tin-cobalt-carbon-containing material, a material (a tin-cobalt-iron-carbon-containing material) that includes tin, cobalt, iron, and carbon as constituent elements may be also preferable. Any composition of the SnCoFeC-containing material may be adopted. To give an example, in a case where a content of iron is set smaller, a content of carbon may be from 9.9 mass % to 29.7 mass % both inclusive, a content of iron may be from 0.3 mass % to 5.9 mass % both inclusive, and a ratio of contents of tin and cobalt (Co/(Sn+Co)) may be from 30 mass % to 70 mass % both inclusive. Alternatively, in a case where the content of iron is set larger, the content of carbon may be from 11.9 mass % to 29.7 mass % both inclusive, the ratio of contents of tin, cobalt, and iron ((Co+Fe)/(Sn+Co+Fe)) may be from 26.4 mass % to 48.5 mass % both inclusive, and the ratio of contents of cobalt and iron (Co/(Co+Fe)) may be from 9.9 mass % to 79.5 mass % both inclusive. Such composition ranges allow for achievement of high energy density. It is to be noted that physical properties (such as a half width) of the tin-cobalt-iron-carbon-containing material are similar to physical properties of the foregoing tin-cobalt-carbon-containing material.

Other than the materials mentioned above, the anode material may be, for example, one or more of materials such as a metal oxide and a polymer compound. Non-limiting examples of the metal oxide may include iron oxide, ruthenium oxide, and molybdenum oxide. Non-limiting examples of the polymer compound may include polyacetylene, polyaniline, and polypyrrole.

Details of the anode binder may be similar to, for example, details of the foregoing cathode binder. Moreover, details of the anode conductor may be similar to, for example, details of the foregoing cathode conductor.

The anode active material layer 22B may be formed by, for example, one or more of a coating method, a vapor-phase method, a liquid-phase method, a spraying method, and a firing method (sintering method). The coating method may be, for example, a method in which, after a particulate (powder) anode active material is mixed with, for example, an anode binder, the mixture is dispersed in a solvent such as an organic solvent, and the resultant is applied onto the anode current collector 22A. Non-limiting examples of the vapor-phase method may include a physical deposition method and a chemical deposition method. More specifically, non-limiting examples thereof may include a vacuum evaporation method, a sputtering method, an ion plating method, a laser ablation method, a thermal chemical vapor deposition method, a chemical vapor deposition (CVD) method, and a plasma chemical vapor deposition method. Non-limiting examples of the liquid-phase method may include an electrolytic plating method and an electroless plating method. The spraying method is a method in which an anode active material in a fused state or a semi-fused state is sprayed to the anode current collector 22A. The firing method may be, for example, a method in which, after the mixture dispersed in, for example, the solvent is applied onto the anode current collector 22A by the coating method, the resultant is subjected to heat treatment at a temperature higher than a melting point of, for example, the anode binder. For example, one or more of firing methods such as an atmosphere firing method, a reactive firing method, and a hot press firing method may be employed as the firing method.

In the secondary battery, as described above, in order to prevent lithium metal from being unintentionally precipitated on a surface of the anode 22 in the middle of charge, the electrochemical equivalent of the anode material that has ability to insert and extract lithium may be preferably larger than the electrochemical equivalent of the cathode. In a case where an open circuit voltage (that is, a battery voltage) in a completely-charged state is 4.25 V or higher, an extraction amount of lithium per unit mass is larger than that in a case where the open circuit voltage is 4.20 V, even if the same cathode active material is used. Hence, amounts of the cathode active material and the anode active material are adjusted in accordance therewith. As a result, high energy density is achieved.

### (Coating Film)

The coating film 22C protects the surface of the anode active material layer 22B through coating the surfaces of the anode active material layer 22B therewith. The coating film 22C suppresses decomposition reaction of the electrolytic solution resulting from reactivity of the anode active material layer 22B (the anode active material), which makes the electrolytic solution resistant to decomposition on the surface of the anode active material layer 22B.

The coating film 22C may be formed on the surface of the anode active material layer 22B by charge-discharge treatment performed after fabrication of the secondary battery. Moreover, heat treatment (aging treatment) under appropriate conditions that is performed after the foregoing charge-discharge treatment makes a state (physical properties) of the anode 22 including the coating film 22C appropriate so as to specifically suppress decomposition reaction of the electrolytic solution. It may be considered that the coating film 22C includes, for example, a reactant (decomposition product) of an unsaturated cyclic carbonate ester to be described later. Details of the charge-discharge treatment, details of the aging treatment, and details of the physical properties of the anode 22 are described later.

It is to be noted that the coating film 22C having been subjected to the aging treatment under the foregoing appropriate conditions is closely packed, tough, and stable, and a thickness of the coating film 22C is sufficiently small. Details of the thickness of the coating film 22C are described later.

### [Separator]

The separator 23 may be provided, for example, between the cathode 21 and the anode 22, as illustrated in FIG. 2. The separator 23 passes lithium ions therethrough while preventing current short circuit that results from contact between the cathode 21 and the anode 22.

More specifically, the separator 23 may include, for example, one or more of porous films such as porous films of a synthetic resin and ceramics. The separator 23 may be a laminated film in which two or more porous films are laminated. Non-limiting examples of the synthetic resin may include polytetrafluoroethylene, polypropylene, and polyethylene.

In particular, the separator 23 may include, for example, the foregoing porous film (a base layer) and a polymer compound layer provided on a single surface or both surfaces of the base layer. This makes it possible to improve adhesibility of the separator 23 with respect to each of the cathode 21 and the anode 22, thereby suppressing deformation of the spirally wound electrode body 20. This makes it possible to suppress decomposition reaction of the electrolytic solution and to suppress liquid leakage of the electrolytic solution with which the base layer is impregnated. Accordingly, even if charge and discharge are repeated, resistance is less prone to increase, and the secondary battery is less prone to swell.

The polymer compound layer may include, for example, a polymer material such as polyvinylidene fluoride, which has high physical strength and is electrochemically stable. Note that the kind of the polymer material is not limited to polyvinylidene fluoride. In order to form the polymer compound layer, for example, the base layer may be coated with a solution prepared by dissolving the polymer material in, for example, an organic solvent, and thereafter, the base layer may be dried. Alternatively, the base layer may be immersed in the solution, and thereafter the base layer may be dried.

The polymer compound layer may include, for example, one or more of insulating particles such as inorganic particles. The kind of the inorganic particles may be, for example, aluminum oxide and aluminum nitride.

### [Electrolytic Solution]

The spirally wound electrode body 20 may be impregnated with the electrolytic solution, as described above.

### (Unsaturated Cyclic Carbonate Ester)

The electrolytic solution includes one or more of unsaturated cyclic carbonate esters. The “unsaturated cyclic carbonate ester” is a generic name of a cyclic carbonate ester having one or more unsaturated carbon-carbon bonds (carbon-carbon double bonds).

More specifically, the unsaturated cyclic carbonate esters may be, for example, respective compounds represented by the following formulas (11) to (13).

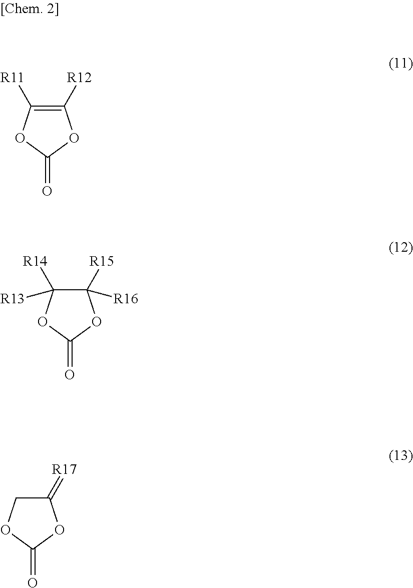

where each of R11 and R12 is one of a hydrogen group and an alkyl group, each of R13 to R16 is one of a hydrogen group, an alkyl group, a vinyl group, and an allyl group, one or more of R13 to R16 are one of the vinyl group and the allyl group, R17 is a group represented by >CR171R172, and each of R171 and R172 is one of a hydrogen group and an alkyl group.

The compound represented by the formula (11) is a vinylene carbonate-based compound. Each of R11 and R12 is not particularly limited, as long as each of R11 and R12 is one of the hydrogen group and the alkyl group, as described above. The number of carbons in the alkyl group is not particularly limited. Specific but non-limiting examples of the alkyl group may include a methyl group, an ethyl group, and a propyl group. R11 and R12 may be groups of a same kind or groups of different kinds. R11 and R12 may be bound to each other.

Specific but non-limiting examples of the vinylene carbonate-based compound may include vinylene carbonate (1,3-dioxol-2-one), methylvinylene carbonate (4-methyl-1,3-dioxol-2-one), ethylvinylene carbonate (4-ethyl-1,3-dioxol-2-one), 4, 5-dimethyl-1,3-dioxol-2-one, and 4,5-diethyl -1,3-dioxol-2-one.

The compound represented by the formula (12) is a vinyl ethylene carbonate-based compound. Each of R13 to R16 is not particularly limited, as long as each of R13 to R16 is the hydrogen group, the alkyl group, the vinyl group, and the allyl group, as described above, on condition that one or more of R13 to R16 are one of the vinyl group and the allyl group. Details of the alkyl group are as described above. R13 to R16 may be groups of a same kind or groups of different kinds. It goes without saying that some of R13 to R16 may be groups of a same kind. Two or more of R13 to R16 may be bound to each other.

Specific but non-limiting examples of the vinyl ethylene carbonate-based compound may include vinyl ethylene carbonate (4-vinyl-1,3-dioxolane-2-one), 4-methyl-4-vinyl-1,3-dioxolane-2-one, 4-ethyl-4-vinyl-1,3-dioxolane-2-one, 4-n-propyl-4-vinyl-1,3-dioxolane-2-one, 5-methyl-4-vinyl -1,3-dioxolane-2-one, 4,4-divinyl-1,3-dioxolane-2-one, and 4,5-divinyl-1,3-dioxolane-2-one.

The compound represented by the formula (13) is a methylene ethylene carbonate-based compound. Each of R171 and R172 is not particularly limited, as long as each of R171 and R172 is one of the hydrogen group and the alkyl group, as described above. It is to be noted that R171 and R172 may be groups of a same kind or groups of different kinds. R171 and R172 may be bound to each other.

Specific but non-limiting examples of the methylene ethylene carbonate-based compound may include methylene ethylene carbonate (4-methylene-1,3-dioxolane-2-one), 4,4-dimethyl-5-methylene-1,3-dioxolane-2-one, and 4,4-diethyl-5-methylene-1,3-dioxolane-2-one.

In addition, non-limiting examples of the unsaturated cyclic carbonate esters may include a catechol carbonate having a benzene ring.

The electrolytic solution includes the unsaturated cyclic carbonate ester, which forms a high-quality coating film 22C (refer to FIG. 2) on a surface of the anode 22 by charge-discharge treatment that is performed after fabrication of the secondary battery, as described later. The “high-quality” relating to the coating film 22C means closely packed, tough, and stable film quality that makes it possible to sufficiently coat the surface of the anode active material layer 22B without impairing the lithium insertion phenomenon and the lithium extraction phenomenon in the anode active material. Accordingly, decomposition reaction of the electrolytic solution on the surface of the anode 22 is suppressed. Hence, even if charge and discharge are repeated, discharge capacity is less prone to decrease, and gas generation resulting from decomposition reaction of the electrolytic solution is less prone to occur.

In particular, the unsaturated cyclic carbonate ester may be preferably the vinylene carbonate-based compound, and more preferably vinylene carbonate, which makes it possible to easily form the high-quality coating film 22C on the surface of the anode 22.

A content of the unsaturated cyclic carbonate ester in the electrolytic solution is not particularly limited, but may be, for example, from 0.01 wt % to 5 wt % both inclusive, which makes it possible to easily form the high-quality coating film 22C on the surface of the anode 22.

The “content of the unsaturated cyclic carbonate ester” described here in a case where the unsaturated cyclic carbonate ester includes two or more kinds of unsaturated cyclic carbonate esters is a total sum of contents of the two or more kinds of unsaturated cyclic carbonate esters.

### (Other Materials)

It is to be noted that the electrolytic solution may include one or more of other materials together with the foregoing unsaturated cyclic carbonate ester. The kind of the other materials is not particularly limited; however, non-limiting examples of the other materials may include a solvent and an electrolyte salt.

### (Solvent)

Non-limiting examples of the solvent may include a nonaqueous solvent (an organic solvent). The solvent may include one or more of solvents. An electrolytic solution including the nonaqueous solvent is a so-called nonaqueous electrolytic solution.

Non-limiting examples of the nonaqueous solvent may include a cyclic carbonate ester, a chain carbonate ester, a lactone, a chain carboxylate ester, and a nitrile (mononitrile), which make it possible to achieve, for example, high battery capacity, superior cycle characteristics, and superior storage characteristics.

Specific but non-limiting examples of the cyclic carbonate ester may include ethylene carbonate, propylene carbonate, and butylene carbonate. Specific but non-limiting examples of the chain carbonate ester may include dimethyl carbonate, diethyl carbonate, ethyl methyl carbonate, and methylpropyl carbonate. Specific but non-limiting examples of the lactone may include y-butyrolactone and y-valerolactone. Specific but non-limiting examples of the chain carboxylate ester may include methyl acetate, ethyl acetate, methyl propionate, ethyl propionate, propyl propionate, methyl butyrate, methyl isobutyrate, methyl trimethylacetate, and ethyl trimethylacetate. Specific but non-limiting examples of the nitrile may include acetonitrile, methoxyacetonitrile, and 3-methoxypropionitrile.

Other than the materials mentioned above, non-limiting examples of the nonaqueous solvent may include 1,2-dimethoxyethane, tetrahydrofuran, 2-methyltetrahydrofuran, tetrahydropyran, 1,3-dioxolane, 4-methyl-1,3-dioxolane, 1,3-dioxane, 1,4-dioxane, N,N-dimethylformamide, N-methylpyrrolidinone, N-methyloxazolidinone, N,N′-dimethylimidazolidinone, nitromethane, nitroethane, sulfolane, trimethyl phosphate, and dimethylsulfoxide. These solvents make it possible to achieve similar advantages.

In particular, the nonaqueous solvent may preferably include one or more of ethylene carbonate, propylene carbonate, dimethyl carbonate, diethyl carbonate, and ethyl methyl carbonate. These materials make it possible to achieve, for example, high battery capacity, superior cycle characteristics, and superior storage characteristics.

In this case, a combination of a high-viscosity (high dielectric constant) solvent (having, for example, specific dielectric constant ε≧30) such as ethylene carbonate and propylene carbonate and a low-viscosity solvent (having, for example, viscosity≦1 mPa·s) such as dimethyl carbonate, ethylmethyl carbonate, and diethyl carbonate may be more preferable. The combination allows for an improvement in the dissociation property of the electrolyte salt and ion mobility.

Moreover, non-limiting examples of the nonaqueous solvent may include a halogenated carbonate ester, a dinitrile compound, a diisocyanate compound, a sulfonate ester, an acid anhydride, and a phosphate ester, which make it possible to further improve chemical stability of the electrolytic solution.

The “halogenated carbonate ester” is a generic name of a carbonate ester including one or more halogen elements as constituent elements. Specific but non-limiting examples of the halogenated carbonate ester may include respective compounds represented by the following formulas (14) and (15).

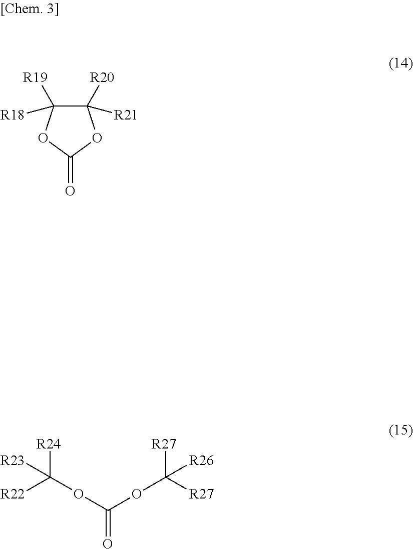

where each of R18 to R21 is one of a hydrogen group, a halogen group, an alkyl group, and a halogenated alkyl group, one or more of R18 to R21 are one of the halogen group and the halogenated alkyl group, each of R22 to R27 is one of a hydrogen group, a halogen group, an alkyl group, and a halogenated alkyl group, and one or more of R22 to R27 are one of the halogen group and the halogenated alkyl group.

The compound represented by the formula (14) is a halogenated cyclic carbonate ester. Each of R18 to R21 is not particularly limited, as long as each of R18 to R21 is one of the hydrogen group, the halogen group, the alkyl group, and the halogenated alkyl group, as described above, under a condition that one or more of R18 to R21 is one of the halogen group and the halogenated alkyl group. It is to be noted that R18 to R21 may be groups of a same kind or groups of different kinds. It goes without saying that some of R18 to R21 may be groups of a same kind. Two or more of R18 to R21 may be bound to each other.

Non-limiting examples of the halogen group may include a fluorine group, a chlorine group, a bromine group, and a iodine group, and the fluorine group may be particularly preferable. The number of the halogen groups may be one or more, and one or more kinds of the halogen groups may be adapted. Details of the alkyl group are as described above. The “halogenated alkyl group” is a generic name of a group in which one or more hydrogen groups in an alkyl group are substituted (halogenated) by a halogen group, and details of the halogen group are as described above.

Specific but non-limiting examples of the halogenated cyclic carbonate ester may include respective compounds represented by the following formulas (14-1) to (14-21), which include geometric isomers. In particular, for example, 4-fluoro-1,3-dioxolane-2-one represented by the formula (14-1) and 4,5-difluoro-1,3-dioxolane-2-one represented by the formula (14-3) may be preferable.

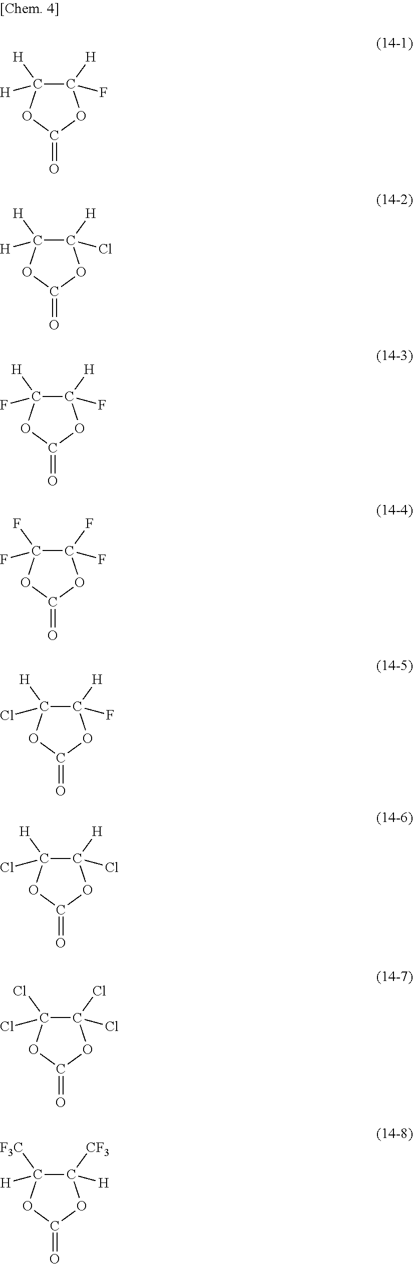

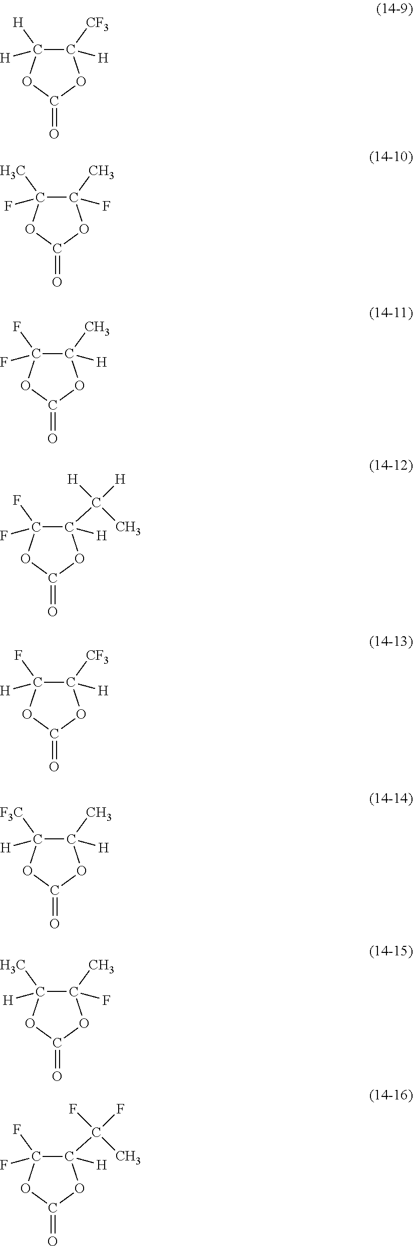

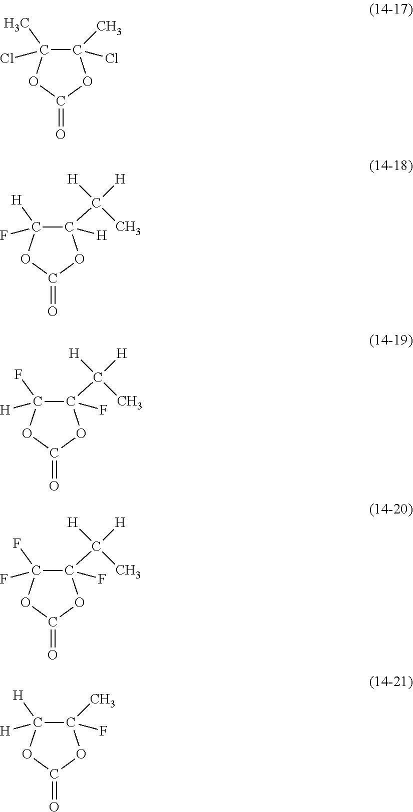

The compound represented by the formula (15) is a halogenated chain carbonate ester. Each of R22 to R27 is not particularly limited, as long as each of R22 to R27 is one of the hydrogen group, the halogen group, the alkyl group, and the halogenated alkyl group, as described above, under a condition that one or more of R22 to R27 is one of the halogen group and the halogenated alkyl group. Details of the halogen group, the alkyl group, and the halogenated alkyl group are as described above. It is to be noted that R22 to R27 may be groups of a same kind or groups of different kinds. It goes without saying that some of R22 to R27 may be groups of a same kind. Two or more of R22 to R27 may be bound to each other.

Specific but non-limiting examples of the halogenated chain carbonate ester may include fluoromethyl methyl carbonate, bis(fluoromethyl) carbonate, and difluoromethyl methyl carbonate.

It is to be noted that a content of the halogenated carbonate ester in the nonaqueous solvent is not particularly limited, but may be, for example, from 0.01 wt % to 10 wt % both inclusive. The “content of the halogenated carbonate ester” described here in a case where the halogenated carbonate ester includes two or more kinds of halogenated carbonate esters is a total sum of contents of the two or more kinds of halogenated carbonate esters.

Non-limiting examples of the dinitrile compound may include a compound represented by the following formula (16). R28 is not particularly limited, as long as R28 is one of an alkylene group and an arylene group. Non-limiting examples of the alkylene group may include a methylene group, an ethylene group, and a propylene group, and non-limiting examples of the arylene group may include a phenylene group and a naphthylene group. The number of carbons in the alkylene group is not particularly limited, but may be, for example, within a range from 1 to 18, and the number of carbons in the arylene group is not particularly limited, but may be, for example, within a range from 6 to 18.

NC—R28-CN (16)

where R28 is one of an alkylene group and an arylene group.

Specific but non-limiting examples of the dinitrile compound may include succinonitrile (NC—C₂H₄—CN), glutaronitrile (NC—C₃H₆—CN), adiponitrile (NC—C₄H₈—CN), sebaconitrile (NC—C₈H₁₀—CN), and phthalonitrile (NC—C₆H₄—CN).

It is to be noted that a content of the dinitrile compound in the nonaqueous solvent is not particularly limited, but may be, for example, within a range from 0.5 wt % to 5 wt % both inclusive.

Non-limiting examples of the diisocyanate compound may include a compound represented by OCN—R29-NCO, where R29 is one of an alkylene group and an arylene group. R29 is not particularly limited, as long as R29 is the alkylene group. Details of the alkylene group may be, for example, as described above. The number of carbons of the alkylene group is not particularly limited, but may be, for example, within a range from 1 to 18. Specific but non-limiting examples of the diisocyanate compound may include OCN—C₆H₁₂—NCO.

It is to be noted that a content of the diisocyanate compound in the nonaqueous solvent is not particularly limited, but may be, for example, within a range from 0.1 wt % to 10 wt % both inclusive.

Non-limiting examples of the sulfonate ester may include a monosulfonate ester and a disulfonate ester.

The monosulfonate ester may be a cyclic monosulfonate ester or a chain monosulfonate ester. Specific but non-limiting examples of the cyclic monosulfonate ester may include sultone such as 1,3-propane sultone and 1,3-propene sultone. Specific but non-limiting examples of the chain monosulfonate ester may include a compound in which a cyclic monosulfonate ester is cleaved at a middle site.

The disulfonate ester may be a cyclic disulfonate ester or a chain disulfonate ester. Specific but non-limiting examples of the cyclic disulfonate ester may include respective compounds represented by formulas (17-1) to (7-3). Specific but non-limiting examples of the chain disulfonate ester may include a compound in which a cyclic disulfonate ester is cleaved at a middle site.

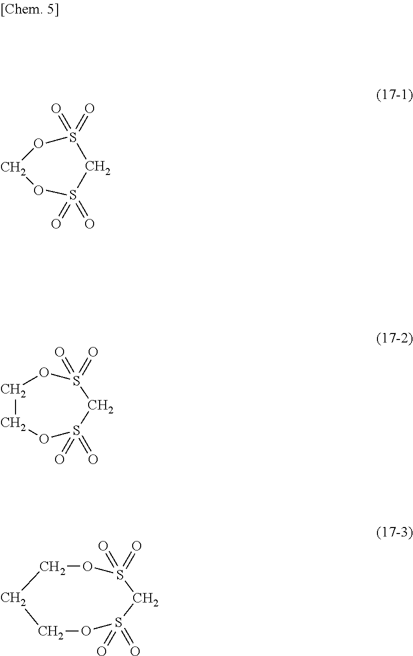 [SMILES: O=S1(=O)CS(=O)(=O)OCO1 ; O=S1(=O)CS(=O)(=O)OCCO1 ; O=S1(=O)CS(=O)(=O)OCCCO1]

It is to be noted that a content of the sulfonate ester in the nonaqueous solvent is not particularly limited, but may be, for example, within a range from 0.01 wt % to 10 wt % both inclusive. The “content of the sulfonate ester” described here in a case where the sulfonate ester includes two or more kinds of sulfonate esters is a total sum of contents of the two or more kinds of sulfonate esters.

Non-limiting examples of the acid anhydride may include a carboxylic anhydride, a disulfonic anhydride, and a carboxylic-sulfonic anhydride.

Specific but non-limiting examples of the carboxylic anhydride may include succinic anhydride, glutaric anhydride, and maleic anhydride. Specific but non-limiting examples of the disulfonic anhydride may include ethanedisulfonic anhydride and propanedisulfonic anhydride. Specific but non-limiting examples of a carboxylic-sulfonic anhydride may include sulfobenzoic anhydride, sulfopropionic anhydride, and sulfobutyric anhydride.

A content of the acid anhydride in the nonaqueous solvent is not particularly limited, but may be, for example, within a range from 0.01 wt % to 10 wt % both inclusive. The “content of the acid anhydride” described here in a case where the acid anhydride includes two or more kinds of acid anhydrides is a total sum of contents of the two or more kinds of acid anhydrides.

Specific but non-limiting examples of the phosphate ester may include trimethyl phosphate, triethyl phosphate, and trialllyl phosphate. It is to be noted that a content of the phosphate ester in the nonaqueous solvent is not particularly limited, but may be, for example, within a range from 0.5 wt % to 5 wt % both inclusive. The “content of the phosphate ester” described here in a case where the phosphate ester includes two or more kinds of phosphate esters is a total sum of contents of the two or more kinds of phosphate esters.

### (Electrolyte Salt)

Non-limiting examples of the electrolyte salt may include one or more of lithium salts. However, the electrolyte salt may include a salt other than the lithium salt. Non-limiting examples of the salt other than lithium may include a salt of a light metal other than lithium.

Specific but non-limiting examples of the lithium salt may include lithium hexafluorophosphate (LiPF₆), lithium tetrafluoroborate (LiBF₄), lithium perchlorate (LiClO₄), lithium hexafluoroarsenate (LiAsF₆), lithium tetraphenylborate (LiB(C₆H₅)₄), lithium methanesulfonate (LiCH₃SO₃), lithium trifluoromethane sulfonate (LiCF₃SO₃), lithium tetrachloroaluminate (LiAlCl₄), dilithium hexafluorosilicate (Li₂SiF₆), lithium chloride (LiCl), and lithium bromide (LiBr).

In particular, one or more of lithium hexafluorophosphate, lithium tetrafluoroborate, lithium perchlorate, and lithium hexafluoroarsenate may be preferable, and lithium hexafluorophosphate may be more preferable. These lithium salts make it possible to decrease the internal resistance.

Moreover, non-limiting examples of the electrolyte salt may include respective compounds represented by the following formulas (31) to (33). It is to be noted that R41 and R43 may be groups of a same kind or groups of different kinds. R51 to R53 may be groups of a same kind or groups of different kinds. It goes without saying that some of R51 to R53 may be groups of a same kind. R61 and R62 may be groups of a same kind or groups of different kinds.

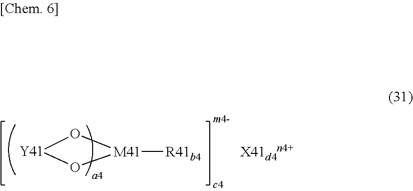

where X41 is one of Group 1 elements and Group 2 elements in the long form of the periodic table of the elements and aluminum (Al), M41 is one of transition metals, and Group 13 elements, Group 14 elements, and Group 15 elements in the long form of the periodic table of the elements, R41 is a halogen group, Y41 is one of —C(═O)—R42—C(═O)—, —C(═O)—CR43₂—, and —C(═O)—C(═O)—, R42 is one of an alkylene group, a halogenated alkylene group, an arylene group, and a halogenated arylene group, R43 is one of an alkyl group, a halogenated alkyl group, an aryl group, and a halogenated aryl group, a4 is an integer of 1 to 4, b4 is an integer of 0, 2, or 4, and each of c4, d4, m4, and n4 is an integer of 1 to 3.

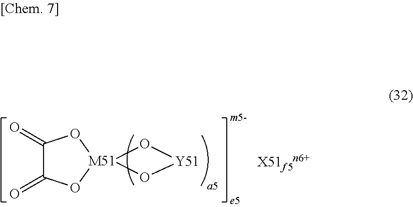

where X51 is one of Group 1 elements and Group 2 elements in the long form of the periodic table of the elements, M51 is one of transition metals, and Group 13 elements, Group 14 elements, and Group 15 elements in the long form of the periodic table of the elements, Y51 is one of —C(═O)—(CR51₂)_{b5}—C(═O)—, —R53₂C—(CR52₂)_{c5}—C(═O)—, —R53₂C—(CR52₂)_{c5}—CR53₂—, —R53₂C—(CR52₂)_{c5}—S(═O)₂—, —S(═O)₂—(CR52₂)_{d5}—S(═O)₂—, and —C(═O)—(CR52₂)_{d5}—S(═O)₂—, each of R51 and R53 is one of a hydrogen group, an alkyl group, a halogen group, and a halogenated alkyl group, one or more of R5's are one of the halogen group and the halogenated alkyl group, one or more of R53's are one of the halogen group and the halogenated alkyl group, R52 is one of a hydrogen group, an alkyl group, a halogen group, and a halogenated alkyl group, each of a5, e5, and n5 is an integer of 1 or 2, each of b5 and d5 is an integer of 1 to 4, c5 is an integer of 0 to 4, and each of f5 and m5 is an integer of 1 to 3.

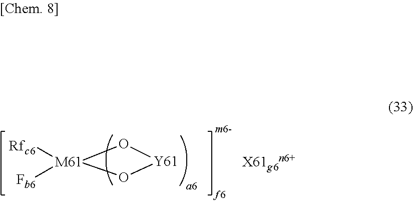

where X61 is one of Group 1 elements and Group 2 elements in the long form of the periodic table of the elements, M61 is one of transition metals, and Group 13 elements, Group 14 elements, and Group 15 elements in the long form of the periodic table of the elements, Rf is one of a fluorinated alkyl group and a fluorinated aryl group, the number of carbons in each of the fluorinated alkyl group and the fluorinated aryl group is from 1 to 10, Y61 is one of —C(═O)—(CR61₂)_{d6}—C(═O)—, —R62₂C—(CR61₂)_{d6}—C(═O)—, —R62₂C—(CR61₂)_{d6}—CR62₂—, —R62₂C—(CR61₂)_{d6}—S(═O)₂—, —S(═O)₂—(CR61₂)ₑ₆—S(═O)₂—, and —C(═O)—(CR61₂)ₑ₆—S(═O)₂—, R61 is one of a hydrogen group, an alkyl group, a halogen group, and a halogenated alkyl group, R62 is one of a hydrogen group, an alkyl group, a halogen group, and a halogenated alkyl group, one or more of R62's are one of the halogen group and the halogenated alkyl group, each of a6, f6, and n6 is an integer of 1 or 2, each of b6, c6, and e6 is an integer of 1 to 4, d6 is an integer of 0 to 4, and each of g6 and m6 is an integer of 1 to 3.

It is to be noted that the Group 1 elements include hydrogen (H), lithium (Li), sodium (Na), potassium (K), rubidium (Rb), cesium (Cs), and francium (Fr). The Group 2 elements include beryllium (Be), magnesium (Mg), calcium (Ca), strontium (Sr), barium (Ba), and radium (Ra). The Group 13 elements include boron (B), aluminum (Al), gallium (Ga), indium (In), and thallium (Tl). The Group 14 elements include carbon (C), silicon (Si), germanium (Ge), tin (Sn), and lead (Pb). The Group 15 elements include nitrogen (N), phosphorus (P), arsenic (As), antimony (Sb), and bismuth (Bi).

Specific but non-limiting examples of the compound represented by the formula (31) may include respective compounds represented by the following formulas (31-1) to (31-6). Specific but non-limiting examples of the compound represented by the formula (32) may include respective compounds represented by the following formulas (32-1) to (32-8). Specific but non-limiting examples of the compound represented by the formula (33) may include a compound represented by the following formula (33-1).

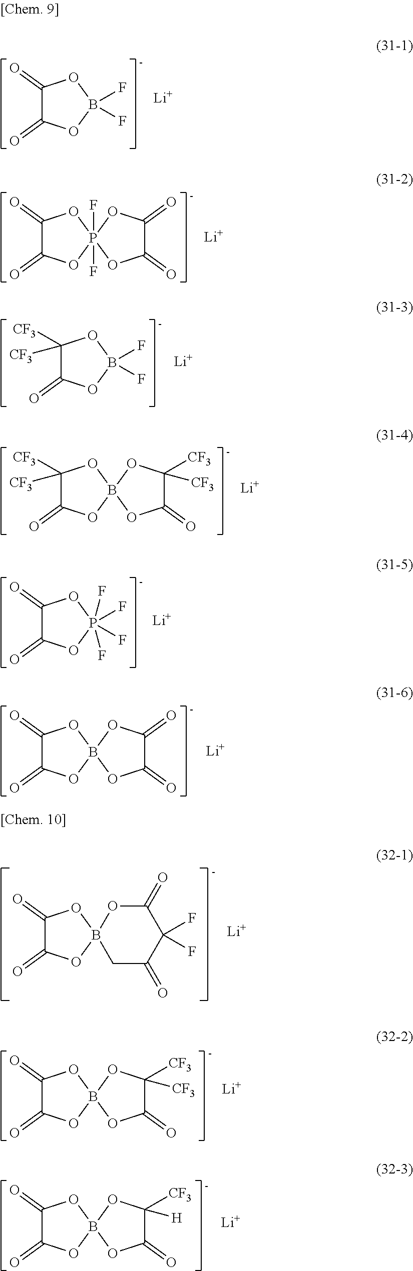

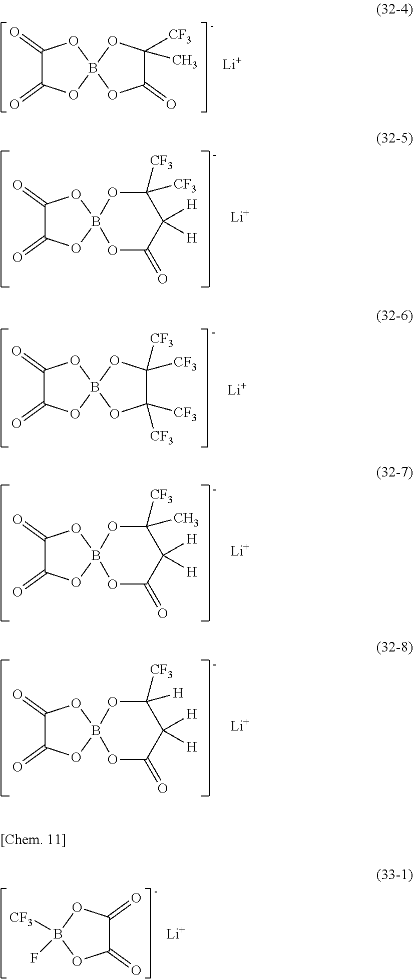

Moreover, the electrolyte salt may be, for example, respective compounds represented by the following formulas (34) to (36). Values of m and n may be the same as or different from each other. Values of p, q, and r may be the same as or different from one another. It goes without saying that the values of two of p, q, and r may be the same as each other.

LiN(CₘF₂ₘ₊₁SO₂)(CₙF₂ₙ₊₁SO₂) (34)

where each of m and n is an integer of 1 or more.

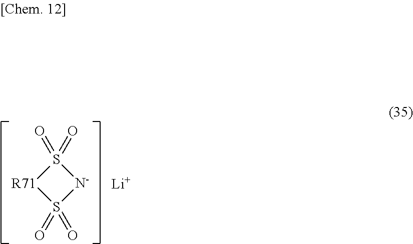

where R71 is a straight-chain perfluoroalkylene group having 2 to 4 carbons or a branched perfluoroalkylene group having 2 to 4 carbons.

LiC(CₚF₂ₚ₋₁SO₂)(C_{q}F_{2q+1}SO₂)(CᵣF₂ᵣ₊₁SO₂) (36)

where each of p, q, and r is an integer of 1 or more.

The compound represented by the formula (34) is a chain imide compound. Specific but non-limiting examples of the chain imide compound may include lithium bis(fluorosulfonyl)imide (LiN(SO₂F)₂), lithium bis(trifluoromethane-sulfonyl)imide (LiN(CF₃SO₂)₂), lithium bis(pentafluoroethanesulfonyl)imide (LiN(C₂F₅SO₂)₂), lithium (trifluoromethanesulfonyl)(pentafluoroethane sulfonyl)imide (LiN(CF₃ SO₂)(C₂F₅ SO₂)), lithium (trifluoromethane sulfonyl)(heptafluoroprop anesulfonyl)imide (LiN(CF₃ SO₂)(C₃F₇SO₂)), and lithium (trifluoromethanesulfonyl)(nonafluorobutanesulfonyl)imide (LiN(CF₃SO₂)(C₄F₉SO₂)).

The compound represented by the formula (35) is a cyclic imide compound. Specific but non-limiting examples of the cyclic imide compound may include respective compounds represented by the following formulas (35-1) to (35-4).

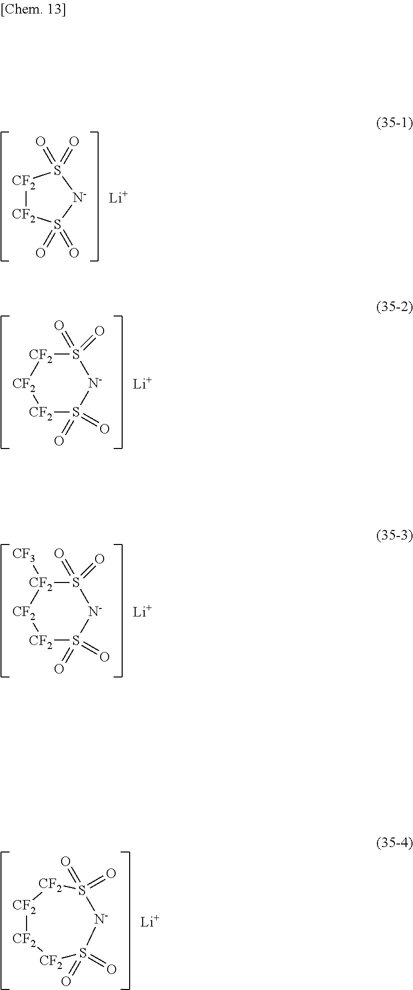

The compound represented by the formula (36) is a chain methide compound. Specific but non-limiting examples of the chain methide compound may include lithium tris(trifluoromethanesulfonyl)methide (LiC(CF₃SO₂)₃).

Moreover, the electrolyte salt may be a phosphorus-fluorine-containing salt such as lithium difluorophosphate (LiPF₂O₂) and lithium fluorophosphate (Li₂PFO₃).

It is to be noted that a content of the electrolyte salt is not particularly limited; however, in particular, the content of the electrolyte salt may be preferably within a range of 0.3 mol/kg to 3.0 mol/kg both inclusive with respect to the solvent. This makes it possible to achieve high ionic conductivity. The “content of the electrolyte salt” described here in a case where the electrolyte salt includes two or more electrolyte salts is a total sum of contents of the two or more electrolyte salts.

### <1-2. Physical Properties of Anode>

Next, description is given of physical properties of the anode 22.

In the secondary battery, in order to achieve superior battery characteristics through specifically suppressing decomposition reaction of the electrolytic solution, physical properties of the anode 22 are made appropriate, as described above.

### [Porosity]

After formation of the coating film 22C, the state of the anode 22 including the coating film 22C is made appropriate by aging treatment under appropriate conditions, as described above. Thus, porosity of the anode 22 is made appropriate.

FIG. 3 illustrates a cross-sectional configuration corresponding to FIG. 2 for description of a procedure of cutting the anode 22. It is to be noted that FIG. 3 illustrates only the anode 22 of the spirally wound electrode body 20 illustrated in FIG. 2.

More specifically, the anode 22 is cut from the surface of the anode 22 (the coating film 22C) to a predetermined depth D (=10 μm), as illustrated in FIG. 3. In this case, a portion (an anode portion 22BP) of the anode active material layer 22B is cut together with a portion (a coating film portion 22CP) of the coating film 22C.

A method of cutting the coating film portion 22CP and the anode portion 22BP is not particularly limited; however, for example, a surface and interfacial cutting analysis system (SAICAS) may be used, as described above. In this case, for example, a cutting range may be 5 mm×5 mm. A total sum (a total thickness) T of a thickness of the coating film portion 22CP and a thickness of the anode portion 22BP may be equal to the foregoing depth D.

Porosity of the anode portion 22BP measured with use of a mercury intrusion technique may be within a range from 30% to 60% both inclusive, and may be preferably within a range from 30% to 50% both inclusive. Hence, decomposition reaction of the electrolytic solution on the surface of the anode 22 is remarkably suppressed, while lithium is smoothly and sufficiently inserted in and extracted from the anode 22. Accordingly, even if charge and discharge are repeated, discharge capacity is hardly decreased, and gas generation hardly occurs, thereby improving the battery characteristics.

More specifically, in a case where after formation of the coating film 22C, the aging treatment is not performed on the anode 22 including the coating film 22C, or the aging treatment is performed on the anode 22 under inappropriate conditions, the state of the coating film 22C remains unstable. Accordingly, if the secondary battery is repeatedly charged and discharged thereafter, a process of breaking down the coating film 22C and thereafter reforming the coating film 22C is repeated.

In this case, every time the coating film 22C is broken down and thereafter reformed, a formation material of the coating film 22C is easily intruded into a plurality of pores present inside the anode active material layer 22B. The plurality of pores are movement (insertion and extraction) paths of lithium during charge and discharge. Accordingly, the plurality of pores are more easily filled with the formation material of material layer 22B. In other words, if the porosity of the anode portion 22BP is measured, the porosity is pronouncedly decreased. Accordingly, lithium is less prone to be inserted in and extracted from the anode 22 by a decrease in the number of the plurality of pores present inside the anode active material layer 22B; therefore, if charge and discharge are repeated, discharge capacity is easily decreased.

In addition, if the process of breaking down the coating film 22C and thereafter reforming the coating film 22C is repeated, the coating film 22C is less prone to suppress decomposition reaction of the electrolytic solution resulting from reactivity of the anode active material layer 22B (the anode active material); therefore, the electrolytic solution is easily decomposed on the surface of the anode active material layer 22B. Accordingly, if charge and discharge are repeated, discharge capacity is decreased more easily, and gas is easily generated.

Hence, in a case where the aging treatment is not performed or in a case where the aging treatment is performed under inappropriate conditions, if charge and discharge are repeated, discharge capacity is easily decreased, and gas is easily generated, which makes it difficult to improve the battery characteristics.

In contrast, in a case where after formation of the coating film 22C, the aging treatment is performed under appropriate conditions, the state of the coating film 22C is stabilized. Accordingly, even if the secondary battery is repeatedly charged and discharged thereafter, the coating film 22C is easily maintained without being broken down.

In this case, the formation material of the coating film 22C is less prone to be intruded into the plurality of pores present inside the anode active material layer 22B. Accordingly, the plurality of pores are less prone to be filled with the formation material of the coating film 22C; therefore, the porosity of the anode active material layer 22B is less prone to be decreased. In other words, if the porosity of the anode portion 22BP is measured, initial (after formation of the secondary battery) porosity is almost maintained, and the porosity is sufficiently large accordingly. Hence, almost maintaining the number of the plurality of pores present inside the anode active material layer 22B makes it easier to insert and extract lithium in the anode 22; therefore, even if charge and discharge are repeated, discharge capacity is less prone to be decreased.

Moreover, the coating film 22C is resistant to breakdown, which makes it easier to suppress decomposition reaction of the electrolytic solution resulting from reactivity of the anode active material layer 22B (the anode active material). Hence, the electrolytic solution is less prone to be decomposed on the surface of the anode active material layer 22B. Accordingly, even if charge and discharge are repeated, discharge capacity is still less prone to be decreased, and the gas generation is less prone to occur.

Accordingly, in a case where the aging treatment is performed under appropriate conditions, even if charge and discharge are repeated, discharge capacity is less prone to be decreased, and gas generation is less prone to occur, which makes it possible to improve the battery characteristics.

It is to be noted that the porosity of the anode portion 22BP is measured to examine porosity of the anode active material layer 22B, because the anode portion 22BP is a portion closer to the coating film 22C of the anode active material layer 22B.

More specifically, the plurality of pores are filled with the formation material of the coating film 22C more easily on side closer to the coating film 22C than on side farther from the coating film 22C inside the anode active material layer 22B. Accordingly, to examine whether use of an appropriate state (physical properties) of the coating film 22C makes it less prone to fill the plurality of pores with the formation material of the coating film 22C, it is more effective to measure porosity on the side closer to the coating film 22C of the anode active material layer 22B than to measure porosity on the side farther from the coating film 22C of the anode active material layer 22B.

The porosity described here may be measured with use of, for example, a mercury porosimeter using the mercury intrusion technique. The state (physical properties) of the coating film 22C exerts a large influence on the porosity of the anode portion 22, i.e., ease of filling the plurality of pores present inside the anode portion 22BP. Accordingly, in a case where the porosity of the anode portion 22BP is measured, the porosity is measured in a state in which the coating film portion 22CP is attached to the anode portion 22BP. In this case, surface tension of mercury is equal to 485 mN/m, a contact angle of mercury is equal to 130°, and a relationship between a pore diameter of a pore and pressure is approximate to 180/pressure=the pore diameter. The mercury porosimeter may be, for example, a mercury porosimeter (AutoPore 9500 series) available from Micromeritics Instrument Corp., located in U.S.A.

In order to reproducibly measure the porosity at high accuracy, before cutting the anode 22, the anode 22 may be preferably subjected to preprocessing.

Through the preprocessing, for example, the anode 22 may be cleaned with use of, for example, an organic solvent to remove the electrolyte salt and any other material remaining inside the plurality of pores, and thereafter the anode 22 may be dried. The kind of the organic solvent is not particularly limited; however, the organic solvent may be one or more of organic solvents such as dimethyl carbonate and acetonitrile. A cleaning method is not particularly limited; however, for example, the anode 22 may be immersed in the organic solvent. Immersion time is not particularly limited, but may be preferably one day, and more preferably two days. A drying method is not particularly limited, but may be vacuum drying. Drying time is not particularly limited, but may be preferably one day, and more preferably three days.

It is to be noted that an environment in which the preprocessing is performed may be, for example, a dry environment in which a dew point is controlled to be −50° C. or less. Alternatively, the environment in which the preprocessing is performed may be, for example, inside of a glovebox in which a total of an oxygen concentration and a water concentration is controlled to be 100 ppm or less, which prevents alternation (for example, oxidation) of the anode 22 resulting from atmospheric exposure.

### [Analysis Result of Anode Using Fourier Transform Infrared Spectroscopy]

In a case where the porosity of the anode portion 22BP is made appropriate, the high-quality coating film 22C is formed by the aging treatment under appropriate conditions, as described above. Accordingly, if the anode 22 (the coating film 22C) is analyzed with use of, for example, Fourier transform infrared spectroscopy (FT-IR), an analysis result to be described below is obtained.

Specifically, in an analysis result of the anode 22 with use of the FT-IR (a horizontal axis indicates wave number (cm⁻¹) and a vertical axis indicates transmittance (%)), a peak is detected in a specific wave number range, while a peak is not detected in a wave number range other than the specific wave number range.

More specifically, a peak is detected in a wave number within a range smaller than 1000 cm⁻¹, and a peak is also detected in a wave number within a range larger than 2000 cm⁻¹. In contrast, a peak is not detected in a wave number within a range from 1000 cm⁻¹ to 2000 cm⁻¹ both inclusive.

In the following, for simplification of description, a range in which the wave number is smaller than 1000 cm⁻¹, a range in which the wave number is larger than 2000 cm⁻¹, and a range in which the wave number is from 1000 cm⁻¹to 2000 cm⁻¹ both inclusive are respectively referred to as a “first range”, a “second range”, and a “third range”.

The foregoing analysis result is obtained by analysis of the anode 22 with use of the FT-IR, because in a case where the titanium-containing compound is used as the anode active material, the state (physical properties) of the coating film 22C is made appropriate, as described above, which specifically suppresses decomposition reaction of the electrolytic solution on the surface of the anode 22 while lithium is smoothly and sufficiently inserted in and extracted from the anode 22.

The analysis result of the anode 22 with use of the FT-IR described here is obtained similarly even in a case where the aging treatment is performed under inappropriate conditions after fabrication of the secondary battery using the titanium-containing compound as the anode active material. However, in a case where the aging treatment is performed under inappropriate conditions after fabrication of the secondary battery using the titanium-containing compound as the anode active material, the state (physical properties) of the coating film 22C is not made appropriate. In this case, in a case where the anode 22 of the completed secondary battery having subjected to the aging treatment is analyzed with use of the FT-IR, a peak is detected in each of the first range, the second range, and the third range similarly to a case where a material other than the titanium-containing compound is used as the foregoing anode active material. Accordingly, it is difficult to sufficiently improve battery characteristics with use of the coating film 22C.

It is to be noted that in the third range, mainly five peaks may be detected. A wave number range in which a first peak is detected may be, for example, from 1030 cm⁻¹ to 1060 cm⁻¹. A wave number range in which a second peak is detected may be, for example, from 1030 cm⁻¹ to 1180 cm⁻¹. A wave number range in which a third peak is detected may be, for example, from 1200 cm⁻¹ to 1300 cm⁻¹. A wave number range in which a fourth peak is detected may be, for example, from 1630 cm⁻¹ to 1650 cm⁻¹. A wave number range in which a fifth peak is detected may be, for example, from 1750 cm⁻¹ to 1790 cm ⁻¹.

In this case, as can be seen from the description that the five peaks “may be detected”, all of the five peaks may be detected, or some (one to four) of the five peak may be detected.

In contrast, in a case where the aging treatment is performed under appropriate conditions after the secondary battery using the titanium-containing compound as the anode active material is fabricated, the state (physical properties) of the coating film 22C is made appropriate. In this case, in a case where the anode 22 in the secondary battery having been subjected to the aging treatment is analyzed with use of the FT-IR, while a peak is detected in each of the first range and the second range, a peak is not detected in the third range. In other words, in the third range, mainly the foregoing five peaks are not detected. This makes it possible to sufficiently improve the battery characteristics with use of the coating film 22C, as described above.

It is to be noted that in a case where whether the peak is detected in the third range is determined, distortion (variation) of a so-called base line is not taken into consideration. More specifically, in order to prevent false detection of a peak resulting from distortion of the base line, for example, a peak having a transmittance (%) of less than 2% is not determined as a peak.

Details of composition of the coating film 22C described here is not sufficiently elucidated. However, if the aging treatment is performed under appropriate conditions in the case where the titanium-containing compound is used as the anode active material, the physical properties of the coating film 22C are specifically made appropriate, as described above. Accordingly, since the coating film 22C includes, for example, a reactant of the titanium-containing compound and the unsaturated cyclic carbonate ester obtained by the aging treatment under appropriate conditions, it may be considered that the composition of the coating film 22C including the reactant, etc. sufficiently decreases reactivity of the anode 22 (reactivity of the electrolytic solution).

In a case where the anode 22 is analyzed with use of the FT-IR, the secondary battery in a predetermined state of charge (SOC) may be preferably used. The predetermined state of charge is achieved by charging and discharging the secondary battery under predetermined conditions, and thereafter charging the secondary battery again. The charged state of the secondary battery (the anode 22) is made uniform to assure reproducibility of an analysis result with use of the FT-IR. Details of charge and discharge conditions and the charged state are described later.

It is to be noted that the analysis result of the anode 22 with use of the foregoing FT-TR, that is, an analysis result that the peak is detected in each of the first range and the second range and the peak is not detected in the third range is a qualitative analysis result. Accordingly, it may be considered that a similar analysis result is obtained independently of differences such as a difference in analysis apparatus and a difference in analysis conditions.

Note that an example of the analysis apparatus and an example of the analysis condition is given for confirmation. As the analysis apparatus, for example, a FTIR spectrometer Cary630 available from Agilent Technologies Japan, Ltd. located in Tokyo, Japan is used. The analysis conditions may be, for example, a spectrum range=4000 cm⁻¹ to 650 cm⁻¹ both inclusive, resolution=2cm⁻¹, a sampling technique=attenuated total reflection (ATR), and detector type=deuterium tri-glycine sulfate (DTGS). The “ATR” relating to the sampling technique is a technique (method) using total internal reflection resulting from an evanescent wave, and enables samples in a solid or liquid state to be analyzed directly without preprocessing. The “DTGS” relating to the detector type is a detector that operates at room temperature, and is suitable for analysis in a wide wave number range (the wave number=7800 cm⁻¹ to 350 cm⁻¹ both inclusive). In particular, the DTGS is superior for analysis of a sample having high transmittance or high reflectivity.

### [Thickness of Coating Film]

The thickness of the coating film 22C formed by the foregoing aging treatment under appropriate conditions may be sufficiently thin. As compared with the case where the aging treatment is not performed and the case where the aging treatment is performed under inappropriate conditions, the state of the coating film 22C is stabilized. In this case, it may be considered that the coating film 22C is homogenized, closely packed, and made tough. Accordingly, the surface of the anode active material layer 22B is sufficiently coated without impairing the lithium insertion phenomenon and the lithium extraction phenomenon in the anode active material.

More specifically, the thickness of the coating film 22C may be, for example, 100 nm or less, and more specifically, within a range from 10 nm to 100 nm both inclusive.

### <1-3. Operation>

Next, description is given of operation of the secondary battery.

The secondary battery may operate as follows, for example. When the secondary battery is charged, lithium ions are extracted from the cathode 21, and the extracted lithium ions are inserted in the anode 22 through the electrolytic solution. In contrast, when the secondary battery is discharged, lithium ions are extracted from the anode 22, and the extracted lithium ions are inserted in the cathode 21 through the electrolytic solution.

### <1-4. Manufacturing Method>

Next, description is given of a method of manufacturing the secondary battery. The secondary battery may be manufactured by the following procedure, for example.

### [Fabrication of Cathode]

In a case where the cathode 21 is fabricated, first, the cathode active material, and, on as-necessary basis, for example, the cathode binder and the cathode conductor may be mixed to obtain a cathode mixture. Subsequently, the cathode mixture may be dispersed in, for example, an organic solvent to obtain paste cathode mixture slurry. Lastly, both surfaces of the cathode current collector 21A may be coated with the cathode mixture slurry, and thereafter, the coated cathode mixture slurry may be dried to form the cathode active material layers 21B. Thereafter, the cathode active material layers 21B may be compression-molded with use of, for example, a roll pressing machine on as-necessary basis. In this case, the cathode active material layers 21B may be heated, or may be compression-molded a plurality of times.

### [Fabrication of Anode]

In a case where the anode 22 is fabricated, the anode active material layers 22B may be formed on both surfaces of the anode current collector 22A by a procedure similar to the foregoing procedure of fabricating the cathode 21. More specifically, the anode active material, and any other material such as the anode binder and the anode conductor may be mixed to obtain an anode mixture. Subsequently, the anode mixture may be dispersed in, for example, an organic solvent to obtain paste anode mixture slurry. Next, both surfaces of the anode current collector 22A may be coated with the anode mixture slurry, and thereafter, the coated anode mixture slurry may be dried to form the anode active material layers 22B. Thereafter, the anode active material layers 22B may be compression-molded with use of, for example, a roll pressing machine on as-necessary basis. It goes without saying that the anode active material layers 22B may be heated, or may be compression-molded a plurality of times.

### [Preparation of Electrolytic Solution]

In a case where the electrolytic solution is prepared, the electrolyte salt may be added to the solvent, and the solvent may be stirred. Accordingly, the electrolyte salt may be dissolved or dispersed in the solvent. Subsequently, the unsaturated cyclic carbonate esters may be added to the solvent including the electrolyte salt, and thereafter, the solvent may be stirred. Accordingly, the unsaturated cyclic carbonate esters may be dispersed in the solvent. The unsaturated cyclic carbonate ester may include one or more kinds of unsaturated cyclic carbonate esters, as described above. Thus, the electrolytic solution including the unsaturated cyclic carbonate ester is prepared.

### [Assembling of Secondary Battery]

In a case where the secondary battery is assembled, the cathode lead 25 may be coupled to the cathode current collector 21A by, for example, a welding method, and the anode lead 26 may be coupled to the anode current collector 22A by, for example, a welding method. Subsequently, the cathode 21 and the anode 22 may be stacked with the separator 23 in between, and the cathode 21, the anode 22, and the separator 23 may be spirally wound to form the spirally wound electrode body 20. Thereafter, the center pin 24 may be inserted in a space provided at the center of the spirally wound electrode body 20.

Subsequently, the spirally wound electrode body 20 may be sandwiched between the pair of insulating plates 12 and 13, and may be contained inside the battery can 11. In this case, an end tip of the cathode lead 25 may be coupled to the safety valve mechanism 15 by, for example, a welding method, and an end tip of the anode lead 26 may be coupled to the battery can 11 by, for example, a welding method. Subsequently, the electrolytic solution may be injected inside the battery can 11, and the spirally wound electrode body 20 may be impregnated with the injected electrolytic solution. Thus, the cathode 21, the anode 22, and the separator 23 may be impregnated with the electrolytic solution.

Lastly, the battery cover 14, the safety valve mechanism 15, and the PTC device 16 may be swaged with the gasket 17 at the open end of the battery can 11. Thus, the secondary battery in a state in which the coating film 22C has not yet been formed is fabricated.

### [Charge-Discharge Treatment]

To stabilize the state of the secondary battery, charge-discharge treatment may be performed on the secondary battery. The “charge-discharge treatment” described here is a process of performing one cycle of charge and discharge on the secondary battery. Charge and discharge conditions are not particularly limited, but may be optionally set in accordance with, for example, the kind of the cathode active material and the kind of the anode active material. More specifically, charge and discharge conditions in a case where the lithium-containing phosphate compound (LiFePO₄) is used as the cathode active material and the lithium-titanium composite oxide (Li₄Ti₅O₁₂) is used as the anode active material may be as follows, for example. In a case where the secondary battery is charged, charge may be performed at a constant current of 0.1 C until the voltage reaches 2.4 V, and thereafter charge may be further performed at a constant voltage of 2.4 V until the current corresponds to 1/30 of an initial current (=0.1 C). In a case where the secondary battery is discharged, discharge may be performed at a constant current of 0.1 C until the voltage reaches 0.5 V. It is to be noted that “0.1 C” refers to a current value at which the battery capacity (theoretical capacity) is completely discharged in 10 hours.

Thus, the coating film 22C may be formed so that the surface of the anode active material layer 22B is coated with the coating film 22C, thereby fabricating the anode 22. Accordingly, a secondary battery in a state in which the coating film 22C is formed is obtained. The coating film 22C is a so-called solid electrolyte interphase (SEI) film, and may include, for example, a reactant of the titanium-containing compound and the unsaturated cyclic carbonate ester, as described above.

### [Aging Treatment]

In a case where the aging treatment is performed on the secondary battery, the secondary battery may be stored in a high temperature environment.

In this case, as described above, in order to make the state (physical properties) of the coating film 22C provided on the surface of the anode active material layer 22B appropriate, the aging treatment may be performed under appropriate conditions. Details of the conditions of the aging treatment are as follows.

A treatment temperature of the aging treatment may be, for example, within a range from 45° C. to 60° C. both inclusive, and may be preferably 45° C.

Treatment time of the aging treatment may be, for example, within a range from 12 hours to 100 hours both inclusive, and may be preferably 48 hours.

A state of charge of the secondary battery in the aging treatment may be, for example, within a range from 25% to 75% both inclusive.

Through the aging treatment, the state (physical properties) of the coating film 22C is made appropriate, as described above; therefore, if the secondary battery is charged and discharged after the aging treatment is performed, the coating film 22C is resistant to breakdown. Thus, the cylindrical secondary battery is completed.

### <1-5. Action and Effects>

According to the cylindrical type secondary battery, the anode 22 includes the titanium-containing compound, the electrolytic solution includes the unsaturated cyclic carbonate ester, and porosity of the anode portion 22BP is within a range from 30% to 50% both inclusive.

In this case, the state (physical properties) of the coating film 22C is made appropriate as described above; therefore, decomposition reaction of the electrolytic solution on the surface of the anode 22 is remarkably suppressed while lithium is smoothly and sufficiently inserted in and extracted from the anode 22. Accordingly, even if charge and discharge are repeated, discharge capacity is hardly decreased, and gas generation hardly occurs, which makes it possible to achieve superior battery characteristics.

In particular, in a case where, through the analysis of the anode 22 (the coating film 22C) with use of the FT-IR, while the peak is detected in each of the first range and the second range, the peak is not detected in the third range, the state (physical properties) of the coating film 22C is made appropriate, which makes it possible to achieve the foregoing effects.

Moreover, the titanium-containing compound includes one or both of the titanium oxide and the lithium-titanium composite oxide, which further suppresses decomposition reaction of the electrolytic solution resulting from reactivity of the anode 22. This makes it possible to achieve a higher effect.

Further, in a case where the unsaturated cyclic carbonate ester includes vinylene carbonate, or the content of the unsaturated cyclic carbonate ester in the electrolytic solution is within a range from 0.01 wt % to 5 wt % both inclusive, the high-quality coating film 22C is easily formed on the surface of the anode 22, which makes it possible to achieve a higher effect.

Furthermore, the thickness of the coating film 22C is 100 nm or less, which makes the state of the coating film 22C homogeneous, closely packed, and tough. Accordingly, the surface of the anode active material layer 22B is sufficiently coated without impairing the lithium insertion phenomenon and the lithium extraction phenomenon in the anode active material, which makes it possible to achieve a higher effect.

In addition, according to the method of manufacturing the cylindrical type secondary battery, the secondary battery including the anode 22 provided with the anode active material layer 22B that includes the titanium-containing compound, and the electrolytic solution including the unsaturated cyclic carbonate ester is fabricated, the charge-discharge treatment is performed on the secondary battery to form the coating film 22C, and thereafter the aging treatment is performed on the secondary under appropriate conditions. This makes it possible to easily and stably manufacture the secondary battery in which the porosity of the anode portion 22BP is within a range from 30% to 50% both inclusive.

### <2. Secondary Battery (Laminated Film Type)>

Next, description is given of another secondary battery according to the embodiment of the present technology.

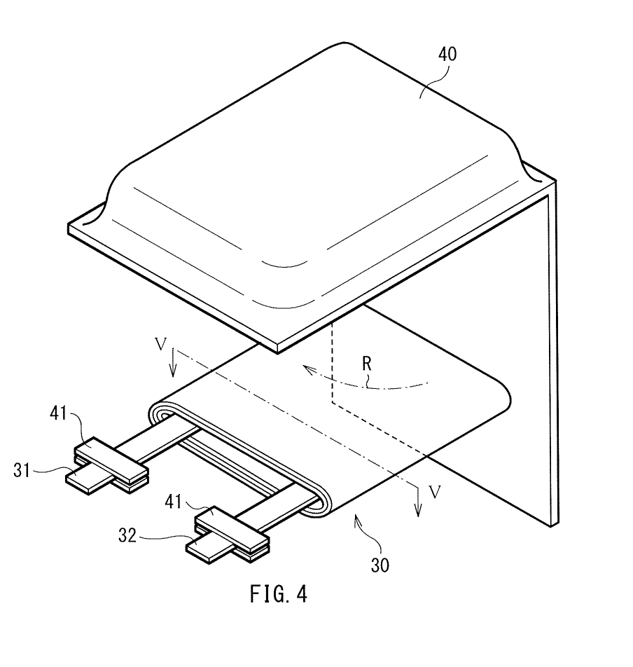

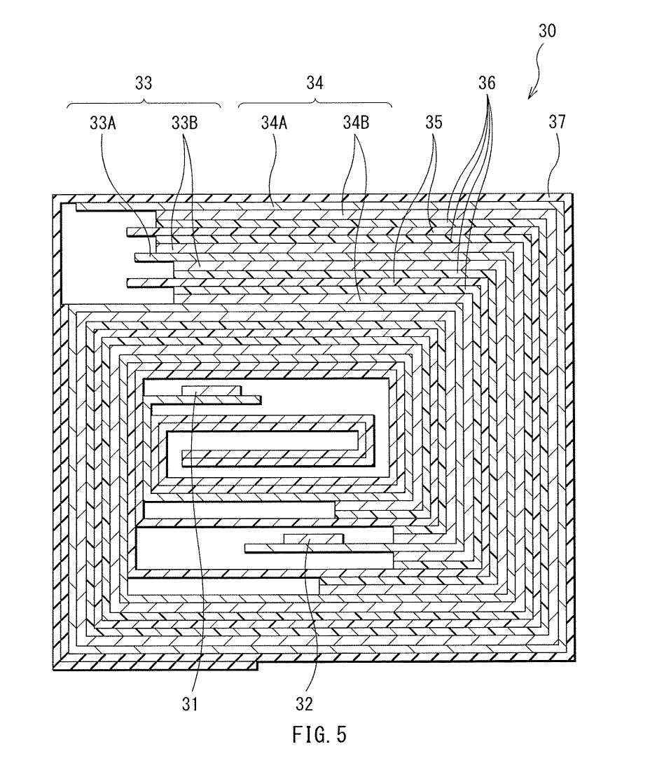

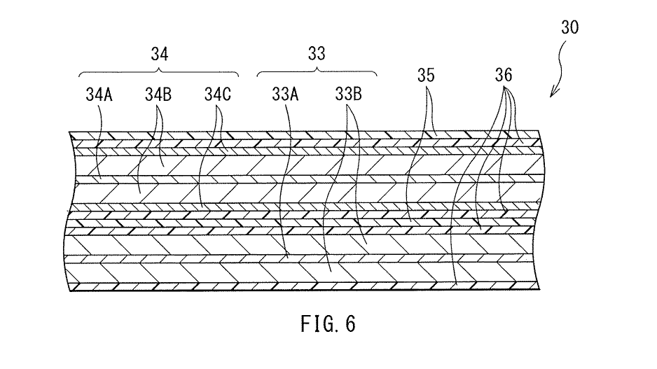

FIG. 4 illustrates a perspective configuration of another secondary battery. FIG. 5 illustrates a cross-sectional configuration taken along a line V-V of a spirally wound electrode body 30 illustrated in FIG. 4. FIG. 6 is an enlarged view of part of the cross-sectional configuration of the spirally wound electrode body 30 illustrated in FIG. 5. It is to be noted that FIG. 4 illustrates a state in which the spirally wound electrode body 30 and an outer package member 40 are separated from each other.

As can be seen from FIG. 4, the secondary battery may be, for example, a so-called laminated film type lithium-ion secondary battery. In following description, the components of the cylindrical type secondary battery that have been already described are used where appropriate.

### <2-1. Configuration>

In the secondary battery, for example, the spirally wound electrode body 30 as a battery element may be contained inside the film-like outer package member 40, as illustrated in FIG. 4. The spirally wound electrode body 30 may be formed as follows, for example. A cathode 33 and an anode 34 may be stacked with a separator 35 and an electrolyte layer 36 in between, and the cathode 33, the anode 34, the separator 35, and the electrolyte layer 36 may be spirally wound to form the spirally wound electrode body 30. An outermost periphery of the spirally wound electrode body 30 may be protected by a protective tape 37. The electrolyte layer 36 may be interposed, for example, between the cathode 33 and the separator 35 and may be interposes, for example, between the anode 34 and the separator 35. A cathode lead 31 may be attached to the cathode 33, and an anode lead 32 may be attached to the anode 34.

Each of the cathode lead 31 and the anode lead 32 may be led out from inside to outside of the outer package member 40, for example. The cathode lead 31 may include, for example, one or more of conductive materials such as aluminum (Al), and the cathode lead 31 may have a thin-plate shape or a mesh shape. The anode lead 32 may include, for example, one or more of conductive materials such as copper (Cu), nickel (Ni), and stainless steel, and the anode lead 32 may have, for example, a shape similar to that of the cathode lead 31.

The outer package member 40 may be, for example, one film that is foldable in a direction of an arrow R illustrated in FIG. 4, and the outer package member 40 may have a depression for containing of the spirally wound electrode body 30 in part thereof. The outer package member 40 may be a laminated film in which a fusion bonding layer, a metal layer, and a surface protective layer are laminated in this order, for example. In a process of manufacturing the secondary battery, the outer package member 40 may be folded so that portions of the fusion-bonding layer face each other with the spirally wound electrode body 30 in between, and thereafter outer edges of the portions of the fusion bonding layer may be fusion-bonded. Alternatively, two laminated films bonded to each other by, for example, an adhesive may form the outer package member 40. The fusion bonding layer may include one or more of films made of polyethylene, polypropylene, and other materials. The metal layer may include, for example, one or more of an aluminum foil and other metal materials. The surface protective layer may include, for example, one or more of films made of nylon, polyethylene terephthalate, and other materials.

In particular, the outer package member 40 may be preferably an aluminum laminated film in which a polyethylene film, an aluminum foil, and a nylon film are laminated in this order. However, the outer package member 40 may be a laminated film having any other laminated structure, a polymer film such as polypropylene, or a metal film.

For example, an adhesive film 41 for prevention of outside air intrusion may be inserted between the outer package member 40 and the cathode lead 31. Moreover, for example, the foregoing adhesive film 41 may be inserted between the outer package member 40 and the anode lead 32. The adhesive film 41 may include a material having adhesibility with respect to the cathode lead 31 and the anode lead 32. Non-limiting examples of the material having adhesibility may include a polyolefin resin. More specifically, the material having adhesibility may include one or more of polyethylene, polypropylene, modified polyethylene, and modified polypropylene.

The cathode 33 may include, for example, a cathode current collector 33A and a cathode active material layer 33B, as illustrated in FIGS. 5 and 6. The anode 34 may include, for example, an anode current collector 34A, an anode active material layer 34B, and a coating film 34C. It is to be noted that the coating film 34C is not illustrated in FIG. 5.

The configurations of the cathode current collector 33A, the cathode active material layer 33B, the anode current collector 34A, the anode active material layer 34B, and the coating film 34C may be similar to, for example, the configurations of the cathode current collector 21A, the cathode active material layer 21B, the anode current collector 22A, the anode active material layer 22B, and the coating film 22C, respectively. The configuration of the separator 35 may be similar to, for example, the configuration of the separator 23.

In other words, the anode 34 may include a titanium-containing compound. Moreover, porosity of a portion corresponding to the anode portion 22BP of the anode active material layer 34B is within a range from 30% to 50% both inclusive.

The electrolyte layer 36 may include an electrolytic solution and a polymer compound. The configuration of the electrolytic solution may be similar to, for example, the configuration of the electrolytic solution used in the foregoing cylindrical type secondary battery. In other words, the electrolytic solution may include an unsaturated cyclic carbonate ester. The electrolyte layer 36 described here may be a so-called gel electrolyte, and the electrolytic solution may be held by the polymer compound. The gel electrolyte achieves high ionic conductivity (for example, 1 mS/cm or more at room temperature), and prevents liquid leakage of the electrolytic solution. It is to be noted that the electrolyte layer 36 may further include one or more of other materials such as an additive.

The polymer material may include, for example, one or more of polyacrylonitrile, polyvinylidene fluoride, polytetrafluoroethylene, polyhexafluoropropylene, polyethylene oxide, polypropylene oxide, polyphosphazene, poly siloxane, polyvinyl fluoride, polyvinyl acetate, polyvinyl alcohol, poly(methyl methacrylate), polyacrylic acid, polymethacrylic acid, styrene-butadiene rubber, nitrile-butadiene rubber, polystyrene, and polycarbonate. In addition thereto, the polymer material may be a copolymer. The copolymer may be, for example, a copolymer of vinylidene fluoride and hexafluoropylene. In particular, polyvinylidene fluoride may be preferable as a homopolymer, and a copolymer of vinylidene fluoride and hexafluoropylene may be preferable as a copolymer. Such polymer compounds are electrochemically stable.

In the electrolyte layer 36 that is a gel electrolyte, the solvent included in the electrolytic solution refers to a wide concept that encompasses not only a liquid material but also a material having ionic conductivity that has ability to dissociate the electrolyte salt. Hence, in a case where a polymer compound having ionic conductivity is used, the polymer compound is also encompassed by the nonaqueous solvent.

It is to be noted that the electrolytic solution may be used instead of the electrolyte layer 36. In this case, the spirally wound electrode body 30 is impregnated with the electrolytic solution.

### <2-2. Operation>

The secondary battery may operate as follows, for example.

When the secondary battery is charged, lithium ions are extracted from the cathode 33, and the extracted lithium ions are inserted in the anode 34 through the electrolyte layer 36. In contrast, when the secondary battery is discharged, lithium ions are extracted from the anode 34, and the extracted lithium ions are inserted in the cathode 33 through the electrolyte layer 36. <2-3. Manufacturing Method>

The secondary battery including the gel electrolyte layer 36 may be manufactured, for example, by one of the following three procedures.

### [First Procedure]

First, the cathode 33 and the anode 34 may be fabricated by a fabrication procedure similar to that of the cathode 21 and the anode 22. More specifically, the cathode 33 may be fabricated by forming the cathode active material layers 33B on both surfaces of the cathode current collector 33A, and the anode 34 may be fabricated by forming the anode active material layers 34B on both surfaces of the anode current collector 34A.

Subsequently, for example, the electrolytic solution, the polymer compound, an organic solvent, etc. may be mixed to prepare a precursor solution. Subsequently, each of the cathode 33 and the anode 34 may be coated with the precursor solution, and the coated precursor solution may be dried to form the gel electrolyte layer 36. Subsequently, the cathode lead 31 may be coupled to the cathode current collector 33A by, for example, a welding method, and the anode lead 32 may be coupled to the anode current collector 34A by, for example, a welding method. Subsequently, the cathode 33 provided with the electrolyte layer 36 and the anode 34 provided with the electrolyte layer 36 may be stacked with the separator 35 in between, and thereafter, the cathode 33, the anode 34, the separator 35, and the electrolyte layers 36 may be spirally wound to fabricate the spirally wound electrode body 30. Thereafter, the protective tape 37 may be attached onto the outermost periphery of the spirally wound body 30.

Subsequently, the outer package member 40 may be folded to interpose the spirally wound electrode body 30, and thereafter, the outer edges of the outer package member 40 may be bonded by, for example, a thermal fusion bonding method to enclose the spirally wound electrode body 30 in the outer package member 40. In this case, the adhesive film 41 may be inserted between the cathode lead 31 and the outer package member 40, and the adhesive film 41 may be inserted between the anode lead 32 and the outer package member 40. Thus, the secondary battery in a state in which the coating film 34C has not yet been formed is obtained.

Subsequently, to stabilize the state of the secondary battery, the charge-discharge treatment may be performed on the secondary battery to form the coating film 34C, thereby fabricating the anode 34. Thus, the secondary battery is fabricated. Charge and discharge conditions are as described above.

Lastly, the aging treatment may be performed on the secondary battery. The details of the aging treatment are as described above. The aging treatment makes the state (physical properties) of the coating film 34C appropriate; therefore, even if the secondary battery is charged and discharged after the aging treatment, the coating film 34C is resistant to breakdown. Thus, the laminated film type secondary battery is completed.

### [Second Procedure]

First, the cathode lead 31 may be coupled to the cathode 33, and the anode lead 32 may be coupled to the anode 34. Subsequently, the cathode 33 and the anode 34 may be stacked with the separator 35 in between and may be spirally wound to fabricate a spirally wound body as a precursor of the spirally wound electrode body 30. Thereafter, the protective tape 37 may be adhered to the outermost periphery of the spirally wound body. Subsequently, the outer package member 40 may be folded to interpose the spirally wound electrode body 30, and thereafter, the outer edges other than one side of the outer package member 40 may be bonded by, for example, a thermal fusion bonding method, and the spirally wound body may be contained inside a pouch formed of the outer package member 40. Subsequently, the electrolytic solution, monomers that are raw materials of the polymer compound, a polymerization initiator, and, on as-necessary basis, other materials such as a polymerization inhibitor may be mixed to prepare a composition for electrolyte. Subsequently, the composition for electrolyte may be injected inside the pouch formed of the outer package member 40. Thereafter, the pouch formed of the outer package member 40 may be hermetically sealed by, for example, a thermal fusion bonding method. Subsequently, the monomers may be thermally polymerized to form the polymer compound. Accordingly, the electrolytic solution may be held by the polymer compound to form the gel electrolyte layer 36. Thus, the secondary battery in a state in which the coating film 34C has not yet been formed may be obtained. Subsequently, to stabilize the state of the secondary battery, the charge-discharge treatment may be performed on the secondary battery to fabricate the coating film 34C, thereby fabricating the anode 34. Lastly, the aging treatment may be performed on the secondary battery to make the state (physical properties) of the coating film 34C appropriate. Thus, the laminated film type secondary battery is completed.

### [Third Procedure]

First, the spirally wound body may be fabricated, and then contained inside the pouch formed of the outer package member 40 in a manner similar to that of the second procedure described above, except that the separator 35 provided with the polymer compound layer is used. Subsequently, the electrolytic solution may be injected inside the pouch formed of the outer package member 40. Thereafter, an opening of the pouch formed of the outer package member 40 may be hermetically sealed by, for example, a thermal fusion bonding method. Subsequently, the resultant may be heated while a weight is applied to the outer package member 40 to cause the separator 35 to be closely attached to the cathode 33 with the polymer compound layer in between and to be closely attached to the anode 34 with the polymer compound layer in between. Through this heating treatment, each of the polymer compound layers may be impregnated with the electrolytic solution, and each of the polymer compound layers may be gelated. Accordingly, the electrolyte layer 36 may be formed. Thus, the secondary battery in a state in which the coating film 34C has not yet been formed may be obtained. Subsequently, to stabilize the state of the secondary battery, the charge-discharge treatment may be performed on the secondary battery to fabricate the anode 34 (the coating film 34C). Lastly, the aging treatment may be performed on the secondary battery to make the state (physical properties) of the coating film 34C appropriate. Thus, the laminated film type secondary battery is completed.

In the third procedure, swollenness of the secondary battery is suppressed more than in the first procedure. Further, in the third procedure, for example, the nonaqueous solvent and the monomers (the raw materials of the polymer compound) are hardly left in the electrolyte layer 36, as compared with the second procedure. Accordingly, the formation process of the polymer compound is favorably controlled. As a result, each of the cathode 33, the anode 34, and the separator 35 is sufficiently and closely attached to the electrolyte layer 36.

### <2-4. Action and Effects>

According to the laminated film type secondary battery, the anode 34 includes the titanium-containing compound, the electrolyte layer 36 (the electrolytic solution) includes the unsaturated cyclic carbonate ester, and the porosity of a portion corresponding to the anode portion 22BP of the anode active material layer 34B is within a range from 30% to 50% both inclusive. Accordingly, even if charge and discharge are repeated, discharge capacity is hardly decreased, and gas generation hardly occurs because of a reason similar to that in the case described in the cylindrical secondary battery, which makes it possible to achieve superior battery characteristics.

According to the method of manufacturing the laminated film type secondary battery, the secondary battery including the anode 34 provided with the anode active material layer 34B that includes the titanium-containing compound, and the electrolyte layer 36 (the electrolytic solution) including the unsaturated cyclic carbonate ester is fabricated, and the charge-discharge treatment is performed on the secondary battery to form the coating film 34C, and thereafter, the aging treatment is performed on the secondary battery under appropriate conditions. This makes it possible to easily and stably manufacture the secondary battery in which the porosity of a portion corresponding to the anode portion 22BP of the anode active material layer 34B is within a range from 30% to 50% both inclusive.

Action and effects other than those described above are similar to those of the cylindrical type secondary battery.

### <3. Applications of Secondary Battery>

Next, description is given of application examples of any of the secondary batteries mentioned above.

Applications of the secondary battery are not particularly limited as long as the secondary battery is applied to, for example, a machine, a device, an instrument, an apparatus, and a system (a collective entity of, for example, a plurality of devices) that are able to use the secondary battery as a driving power source, an electric power storage source for electric power accumulation, or any other source. The secondary battery used as the power source may be a main power source or an auxiliary power source. The main power source is a power source used preferentially irrespective of presence or absence of any other power source. The auxiliary power source may be a power source used instead of the main power source or used being switched from the main power source on as-necessary basis. In a case where the secondary battery is used as the auxiliary power source, the kind of the main power source is not limited to the secondary battery.

Examples of the applications of the secondary battery may include electronic apparatuses (including portable electronic apparatuses) such as a video camcorder, a digital still camera, a mobile phone, a notebook personal computer, a cordless phone, a headphone stereo, a portable radio, a portable television, and a portable information terminal. Further examples thereof may include: a mobile lifestyle appliance such as an electric shaver; a storage device such as a backup power source and a memory card; an electric power tool such as an electric drill and an electric saw; a battery pack used as an attachable and detachable power source of, for example, a notebook personal computer; a medical electronic apparatus such as a pacemaker and a hearing aid; an electric vehicle such as an electric automobile (including a hybrid automobile); and an electric power storage system such as a home battery system for accumulation of electric power for, for example, emergency. It goes without saying that the secondary battery may be employed for an application other than the applications mentioned above.

In particular, the secondary battery may be effectively applicable to, for example, the battery pack, the electric vehicle, the electric power storage system, the electric power tool, and the electronic apparatus. In these applications, superior battery characteristics are demanded, and using the secondary battery of any of the embodiments of the present technology makes it possible to effectively improve performance. It is to be noted that the battery pack is a power source that uses the secondary battery, and may be, for example, a single battery and an assembled battery that are to be described later. The electric vehicle is a vehicle that operates (runs) using the secondary battery as a driving power source, and may be an automobile (such as a hybrid automobile) that includes together a drive source other than the secondary battery, as described above. The electric power storage system is a system that uses the secondary battery as an electric power storage source. For example, in a home electric power storage system, electric power is accumulated in the secondary battery that is the electric power storage source, which makes it possible to use, for example, home electric products with use of the accumulated electric power. The electric power tool is a tool in which a movable section (such as a drill) is allowed to be moved with use of the secondary battery as a driving power source. The electronic apparatus is an apparatus that executes various functions with use of the secondary battery as a driving power source (an electric power supply source).

Hereinafter, specific description is given of some application examples of the secondary battery. It is to be noted that configurations of the respective application examples described below are mere examples, and may be changed as appropriate.

### <3-1. Battery Pack (Single Battery)>

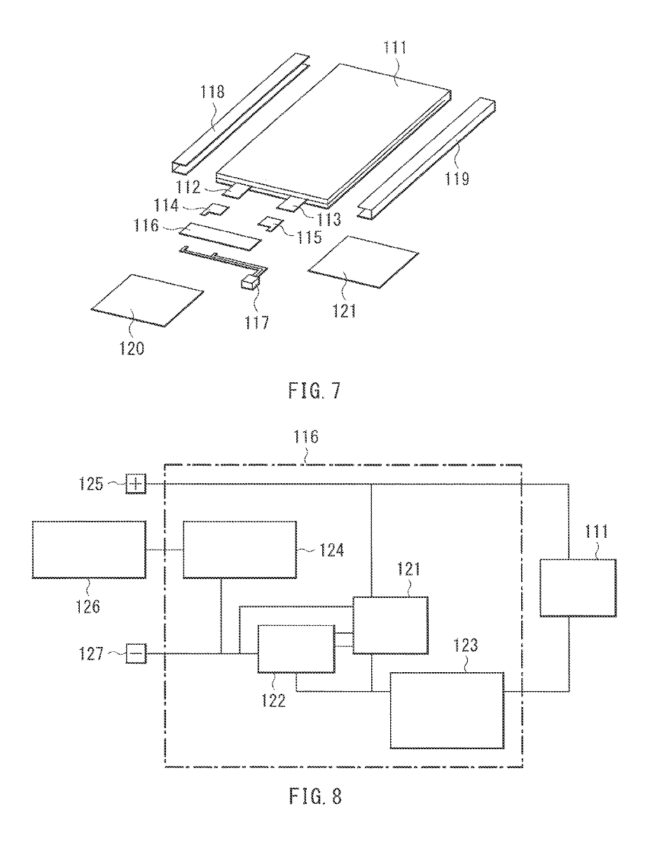

FIG. 7 illustrates a perspective configuration of a battery pack using a single battery. FIG. 8 illustrates a block configuration of the battery pack illustrated in FIG. 7. It is to be noted that FIG. 7 illustrates the battery back in an exploded state.

The battery back described here is a simple battery pack using one secondary battery (a so-called soft pack), and may be mounted in, for example, an electronic apparatus typified by a smartphone. For example, the battery pack may include a power source 111 that is the laminated film type secondary battery, and a circuit board 116 coupled to the power source 111, as illustrated in FIG. 7. A cathode lead 112 and an anode lead 113 may be attached to the power source 111.

A pair of adhesive tapes 118 and 119 may be adhered to both side surfaces of the power source 111. A protection circuit module (PCM) may be formed in the circuit board 116. The circuit board 116 may be coupled to the cathode lead 112 through a tab 114, and be coupled to the anode lead 113 through a tab 115. Moreover, the circuit board 116 may be coupled to a lead 117 provided with a connector for external connection. It is to be noted that while the circuit board 116 is coupled to the power source 111, the circuit board 116 may be protected from upper side and lower side by a label 120 and an insulating sheet 121. The label 120 may be adhered to fix, for example, the circuit board 116 and the insulating sheet 121.

Moreover, for example, the battery pack may include the power source 111 and the circuit board 116 as illustrated in FIG. 8. The circuit board 116 may include, for example, a controller 121, a switch section 122, a PTC device 123, and a temperature detector 124. The power source 111 may be connectable to outside through a cathode terminal 125 and an anode terminal 127, and may be thereby charged and discharged through the cathode terminal 125 and the anode terminal 127. The temperature detector 124 may detect a temperature with use of a temperature detection terminal (a so-called T terminal) 126.

The controller 121 controls an operation of the entire battery pack (including a used state of the power source 111), and may include, for example, a central processing unit (CPU) and a memory.

For example, in a case where a battery voltage reaches an overcharge detection voltage, the controller 121 may so cause the switch section 122 to be disconnected that a charge current does not flow into a current path of the power source 111. Moreover, for example, in a case where a large current flows during charge, the controller 121 may cause the switch section 122 to be disconnected, thereby blocking the charge current.

In contrast, for example, in a case where the battery voltage reaches an overdischarge detection voltage, the controller 121 may so cause the switch section 122 to be disconnected that a discharge current does not flow into the current path of the power source 111. Moreover, for example, in a case where a large current flows during discharge, the controller 121 may cause the switch section 122 to be disconnected, thereby blocking the discharge current.

It is to be noted that the overcharge detection voltage of the secondary battery is not particularly limited, but may be, for example, 4.20 V±0.05 V, and the overdischarge detection voltage is not particularly limited, but may be, for example, 2.4 V±0.1 V.

The switch section 122 switches the used state of the power source 111 (whether the power source 111 is connectable to an external device) in accordance with an instruction from the controller 121. The switch section 122 may include, for example, a charge control switch and a discharge control switch. The charge control switch and the discharge control switch each may be, for example, a semiconductor switch such as a field-effect transistor using a metal oxide semiconductor (MOSFET). It is to be noted that the charge current and the discharge current may be detected on the basis of on-resistance of the switch section 122.

The temperature detector 124 measures a temperature of the power source 111, and outputs a result of the measurement to the controller 121. The temperature detector 124 may include, for example, a temperature detecting element such as a thermistor. It is to be noted that the result of the measurement by the temperature detector 124 may be used, for example, in a case where the controller 121 performs charge and discharge control at the time of abnormal heat generation and in a case where the controller 121 performs a correction process at the time of calculating remaining capacity.

It is to be noted that the circuit board 116 may not include the PTC device 123. In this case, a PTC device may be separately attached to the circuit board 116.

### <3-2. Battery Pack (Assembled Battery)>

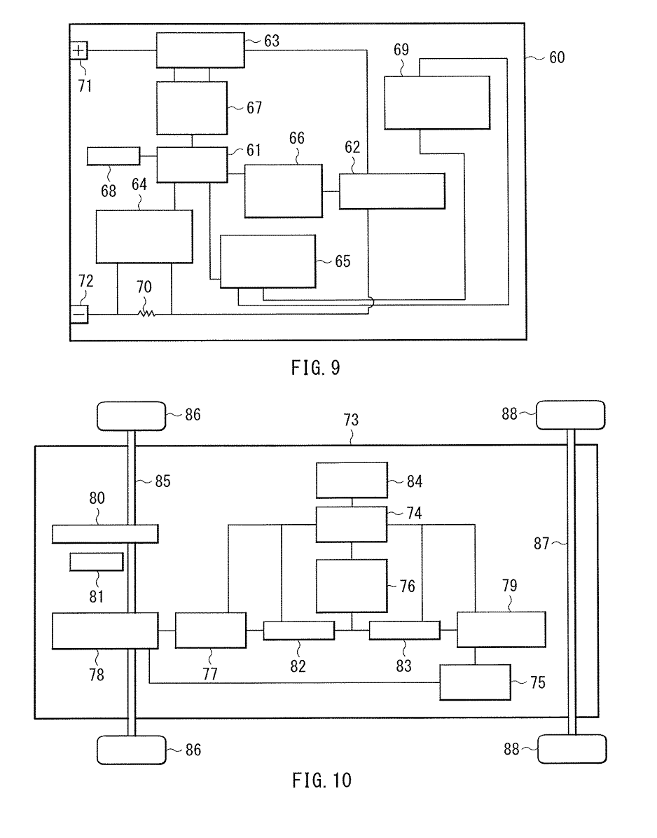

FIG. 9 illustrates a block configuration of a battery pack using an assembled battery.

For example, the battery pack may include a controller 61, a power source 62, a switch section 63, a current measurement section 64, a temperature detector 65, a voltage detector 66, a switch controller 67, a memory 68, a temperature detecting element 69, a current detection resistance 70, a cathode terminal 71, and an anode terminal 72 inside a housing 60. The housing 60 may be made of, for example, a plastic material.

The controller 61 controls an operation of the entire battery pack (including a used state of the power source 62). The controller 61 may include, for example, a CPU. The power source 62 may be, for example, an assembled battery that includes two or more secondary batteries. The secondary batteries may be connected in series, in parallel, or in series-parallel combination. To give an example, the power source 62 may include six secondary batteries in which two sets of series-connected three batteries are connected in parallel to each other.

The switch section 63 switches the used state of the power source 62 (whether the power source 62 is connectable to an external device) in accordance with an instruction from the controller 61. The switch section 63 may include, for example, a charge control switch, a discharge control switch, a charging diode, and a discharging diode. The charge control switch and the discharge control switch each may be, for example, a semiconductor switch such as a field-effect transistor that uses a metal oxide semiconductor (a MOSFET).

The current measurement section 64 measures a current with use of the current detection resistance 70, and outputs a result of the measurement to the controller 61. The temperature detector 65 measures a temperature with use of the temperature detecting element 69, and outputs a result of the measurement to the controller 61. The result of the temperature measurement may be used, for example, in a case where the controller 61 performs charge and discharge control at the time of abnormal heat generation and in a case where the controller 61 performs a correction process at the time of calculating remaining capacity. The voltage detector 66 measures voltages of the secondary batteries in the power source 62, performs analog-to-digital conversion on the measured voltage, and supplies the resultant to the controller 61.

The switch controller 67 controls an operation of the switch section 63 in accordance with signals inputted from the current measurement section 64 and the voltage detector 66.

For example, in a case where a battery voltage reaches an overcharge detection voltage, the switch controller 67 may so cause the switch section 63 (the charge control switch) to be disconnected that a charge current does not flow into a current path of the power source 62. This makes it possible to perform only discharge through the discharging diode in the power source 62. It is to be noted that, for example, when a large current flows during charge, the switch controller 67 may block the charge current.

Further, for example, in a case where the battery voltage reaches an overdischarge detection voltage, the switch controller 67 may so cause the switch section 63 (the discharge control switch) to be disconnected that a discharge current does not flow into the current path of the power source 62. This makes it possible to perform only charge through the charging diode in the power source 62. It is to be noted that, for example, when a large current flows during discharge, the switch controller 67 may block the discharge current.

It is to be noted that the overcharge detection voltage of the secondary battery is not particularly limited, but may be, for example, 4.20 V±0.05 V, and the overdischarge detection voltage is not particularly limited, but may be, for example, 2.4 V±0.1 V.

The memory 68 may include, for example, an EEPROM that is a non-volatile memory. The memory 68 may hold, for example, numerical values calculated by the controller 61 and information of the secondary battery measured in a manufacturing process (such as internal resistance in an initial state). It is to be noted that, in a case where the memory 68 holds full charge capacity of the secondary battery, the controller 61 is allowed to comprehend information such as remaining capacity.

The temperature detecting element 69 measures a temperature of the power source 62, and outputs a result of the measurement to the controller 61. The temperature detecting element 69 may include, for example, a thermistor.

The cathode terminal 71 and the anode terminal 72 are terminals that may be coupled to, for example, an external device (such as a notebook personal computer) driven with use of the battery pack or an external device (such as a battery charger) used for charge of the battery pack. The power source 62 is charged and discharged via the cathode terminal 71 and the anode terminal 72.

### <3-3. Electric Vehicle>

FIG. 10 illustrates a block configuration of a hybrid automobile that is an example of an electric vehicle.

The electric vehicle may include, for example, a controller 74, an engine 75, a power source 76, a driving motor 77, a differential 78, an electric generator 79, a transmission 80, a clutch 81, inverters 82 and 83, and various sensors 84 inside a housing 73 made of metal. Other than the components mentioned above, the electric vehicle may include, for example, a front drive shaft 85 and a front tire 86 that are coupled to the differential 78 and the transmission 80, and a rear drive shaft 87, and a rear tire 88.

The electric vehicle may be runnable with use of one of the engine 75 and the motor 77 as a drive source, for example. The engine 75 is a main power source, and may be, for example, a petrol engine. In a case where the engine 75 is used as the power source, drive power (torque) of the engine 75 may be transferred to the front tire 86 or the rear tire 88 via the differential 78, the transmission 80, and the clutch 81 that are drive sections, for example. It is to be noted that the torque of the engine 75 may be also transferred to the electric generator 79. With use of the torque, the electric generator 79 generates alternating-current electric power. The generated alternating-current electric power is converted into direct-current electric power via the inverter 83, and the converted electric power is accumulated in the power source 76. In a case where the motor 77 that is a conversion section is used as the power source, electric power (direct-current electric power) supplied from the power source 76 is converted into alternating-current electric power via the inverter 82, and the motor 77 is driven with use of the alternating-current electric power. Drive power (torque) obtained by converting the electric power by the motor 77 may be transferred to the front tire 86 or the rear tire 8 via the differential 78, the transmission 80, and the clutch 81 that are the drive sections, for example.

It is to be noted that in a case where speed of the electric vehicle is reduced by a brake mechanism, resistance at the time of speed reduction may be transferred to the motor 77 as torque, and the motor 77 may generate alternating-current electric power by utilizing the torque. It may be preferable that this alternating-current electric power be converted into direct-current electric power via the inverter 82, and the direct-current regenerative electric power be accumulated in the power source 76.

The controller 74 controls an operation of the entire electric vehicle, and may include, for example, a CPU. The power source 76 includes one or more secondary batteries. The power source 76 may be coupled to an external power source, and the power source 76 may be allowed to accumulate electric power by receiving electric power supply from the external power source. The various sensors 84 may be used, for example, for control of the number of revolutions of the engine 75 and for control of an opening level (a throttle opening level) of an unillustrated throttle valve. The various sensors 84 may include, for example, a speed sensor, an acceleration sensor, and an engine frequency sensor.

It is to be noted that, although the description has been given with reference to an example in which the electric vehicle is the hybrid automobile, the electric vehicle may be a vehicle (an electric automobile) that operates with use of only the power source 76 and the motor 77 and without using the engine 75.

### <3-4. Electric Power Storage System>

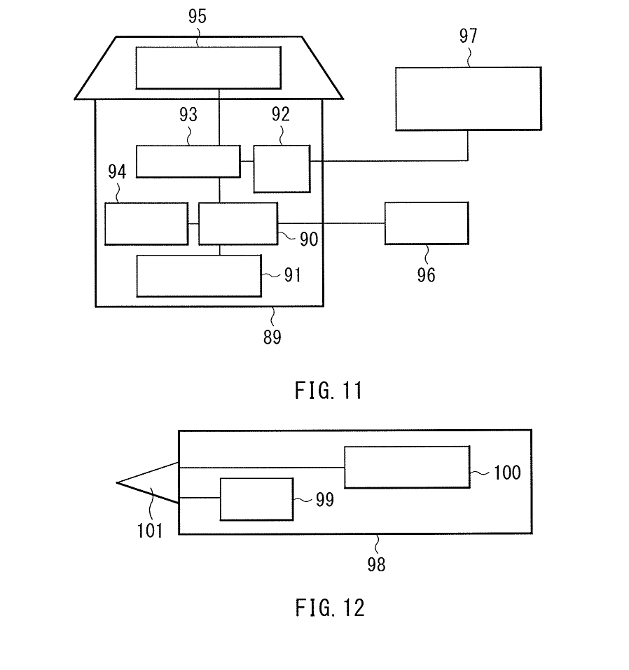

FIG. 11 illustrates a block configuration of an electric power storage system.

The electric power storage system may include, for example, a controller 90, a power source 91, a smart meter 92, and a power hub 93 inside a house 89 such as a general residence or a commercial building.

In this example, the power source 91 may be coupled to an electric device 94 provided inside the house 89 and may be allowed to be coupled to an electric vehicle 96 parked outside the house 89, for example. Further, for example, the power source 91 may be coupled to a private power generator 95 provided in the house 89 via the power hub 93, and may be allowed to be coupled to an outside concentrating electric power system 97 via the smart meter 92 and the power hub 93.

It is to be noted that the electric device 94 may include, for example, one or more home electric products. Non-limiting examples of the home electric products may include a refrigerator, an air conditioner, a television, and a water heater. The private power generator 95 may include, for example, one or more of a solar power generator, a wind power generator, and other power generators. The electric vehicle 96 may include, for example, one or more of an electric automobile, an electric motorcycle, a hybrid automobile, and other electric vehicles. The concentrating electric power system 97 may include, for example, one or more of a thermal power plant, an atomic power plant, a hydraulic power plant, a wind power plant, and other power plants.

The controller 90 controls an operation of the entire electric power storage system (including a used state of the power source 91 ), and may include, for example, a CPU. The power source 91 includes one or more secondary batteries. The smart meter 92 may be an electric power meter that is compatible with a network and is provided in the house 89 demanding electric power, and may be communicable with an electric power supplier, for example. Accordingly, for example, while the smart meter 92 communicates with outside, the smart meter 92 controls balance between supply and demand in the house 89, which allows for effective and stable energy supply.

In the electric power storage system, for example, electric power may be accumulated in the power source 91 from the concentrating electric power system 97, that is an external power source, via the smart meter 92 and the power hub 93, and electric power may be accumulated in the power source 91 from the private power generator 95, that is an independent power source, via the power hub 93. The electric power accumulated in the power source 91 is supplied to the electric device 94 and the electric vehicle 96 in accordance with an instruction from the controller 90. This allows the electric device 94 to be operable, and allows the electric vehicle 96 to be chargeable. In other words, the electric power storage system is a system that makes it possible to accumulate and supply electric power in the house 89 with use of the power source 91.

The electric power accumulated in the power source 91 is allowed to be utilized optionally. Hence, for example, electric power may be accumulated in the power source 91 from the concentrating electric power system 97 in the middle of night when an electric rate is inexpensive, and the electric power accumulated in the power source 91 may be used during daytime hours when the electric rate is expensive.

It is to be noted that the foregoing electric power storage system may be provided for each household (each family unit), or may be provided for a plurality of households (a plurality of family units).

Moreover, the electric power storage system may be applied not only to the consumer applications such as the foregoing general residence but also to commercial applications such as the concentrating electric power system 97, i.e., an electric power supply source typified by a thermal power plant, an atomic power plant, a hydraulic power plant, and a wind power plant. More specifically, description has been given with reference to the case where the electric power storage system is applied to household applications; however, the electric power storage system may be applied to, for example, industrial applications such as an electric power network for grid-connected power (so-called grid) to be used as an electric storage apparatus.

### <3-5. Electric Power Tool>

FIG. 12 illustrates a block configuration of an electric power tool.

The electric power tool described here may be, for example, an electric drill. The electric power tool may include a controller 99 and a power source 100 inside a tool body 98, for example. A drill section 101 that is a movable section may be attached to the tool body 98 in an operable (rotatable) manner, for example.

The tool body 98 may include, for example, a plastic material. The controller 99 controls an operation of the entire electric power tool (including a used state of the power source 100 ), and may include, for example, a CPU. The power source 100 includes one or more secondary batteries. The controller 99 allows electric power to be supplied from the power source 100 to the drill section 101 in accordance with an operation by an operation switch.

### EXAMPLES

Description is given of examples of the present technology.

### (Experimental Examples 1-1 to 1-16)

The secondary batteries (lithium-ion secondary batteries) were fabricated, and thereafter, battery characteristics of the secondary batteries were evaluated.

### [Fabrication of Laminated Film Type Secondary Battery]

Each of laminated film type secondary batteries illustrated in FIGS. 4 to 6 was fabricated by a procedure described below.

The cathode 33 was fabricated as follows. First, 91 parts by mass of a cathode active material (LiFePO₄ that was a lithium-containing phosphate compound), 3 parts by mass of a cathode binder (polyvinylidene fluoride), and 6 part by mass of a cathode conductor (graphite) were mixed to obtain a cathode mixture. Subsequently, the cathode mixture was put into an organic solvent (N-methyl-2-pyrrolidone), and thereafter, the organic solvent was stirred to obtain paste cathode mixture slurry. Subsequently, both surfaces of the cathode current collector 33A (a strip-shaped aluminum foil having a thickness of 12 μm) were coated with the cathode mixture slurry with use of a coating apparatus, and thereafter, the cathode mixture slurry was dried to form the cathode active material layers 33B. Lastly, the cathode active material layers 33B were compression-molded with use of a roll pressing machine. In this case, volume density of the cathode active material layers 33B was 1.7 g/cm³.

The anode 34 was fabricated as follows. First, 90 parts by mass of an anode active material (Li₄Ti₅O₁₂ that was a lithium-titanium composite oxide), 5 parts by mass of an anode binder (polyvinylidene fluoride), and 5 parts by mass of an anode conductor (graphite) were mixed to obtain an anode mixture. Subsequently, the anode mixture was put into an organic solvent (N-methyl-2-pyrrolidone), and thereafter, the organic solvent was stirred to obtain paste anode mixture slurry. Subsequently, both surfaces of the anode current collector 34A (a strip-shaped copper foil having a thickness of 15 μm) were coated with the anode mixture slurry, and thereafter, the anode mixture slurry was dried to form the anode active material layers 34B. Lastly, the anode active material layers 34B were compression-molded with use of a roll pressing machine. In this case, the volume density of the anode active material layers 34B was 1.7 g/cm³.

The electrolytic solution was prepared as follows. An electrolyte salt (LiPF₆) was added into a solvent (propylene carbonate, ethyl methyl carbonate, and dimethyl carbonate), and the solvent was stirred. Thereafter, an unsaturated cyclic carbonate ester (vinylene carbonate (VC) that was a vinylene carbonate-based compound) was further added into the solvent, and the solvent was stirred. In this case, a mixture ratio (weight ratio) of the solvent was propylene carbonate:ethyl methyl carbonate:dimethyl carbonate=40:30:30, and a content of the electrolyte salt was 1 mol/kg with respect to the solvent. A content of the unsaturated cyclic carbonate ester in the electrolytic solution is as illustrated in Table 1.

It is to be noted that, for comparison, an electrolytic solution was prepared in a similar procedure, except that the unsaturated cyclic carbonate ester was not used. The presence or absence of the unsaturated cyclic carbonate ester is as illustrated in Table 1.

The secondary battery was assembled as follows. First, the cathode lead 31 made of aluminum was attached to the cathode current collector 33A by welding, and the anode lead 32 made of copper was attached to the anode current collector 34A by welding. Subsequently, the cathode 33 and the anode 34 were stacked with the separator 35 (a microporous polyethylene film having a thickness of 12 μm) in between to obtain a laminated body. Subsequently, the laminated body was spirally wound in a longitudinal direction, and the protective tape 37 was attached onto the outermost periphery of the laminated body to fabricate the spirally wound electrode body 30. Subsequently, the outer package member 40 was folded to interpose the spirally wound electrode body 30, and thereafter, the outer edges on three sides of the outer package member 40 were thermally fusion-bonded to form a pouch. The outer package member 40 used here was an aluminum laminated film in which a nylon film (having a thickness of 25 μm), an aluminum foil (having a thickness of 40 μm), and a polypropylene film (having a thickness of 30 μm) were laminated in this order from outside. In this case, the adhesive film 41 was inserted between the cathode lead 31 and the outer package member 40, and the adhesive film 41 was inserted between the anode lead 32 and the outer package member 40. Lastly, the electrolytic solution was injected inside the pouch formed of the outer package member 40, and the spirally wound electrode body 30 was impregnated with the electrolytic solution. Thereafter, outer edges on the remaining one side of the outer package member 40 were thermally fusion-bonded in a reduced-pressure environment. Thus, the spirally wound electrode body 30 was sealed inside the outer package member 40 to obtain each of the secondary batteries in which the coating film 34C had not yet been formed.

In a case where the charge-discharge treatment was performed on the secondary batteries, the secondary batteries were charged and discharged. Accordingly, the coating film 34C was formed on the surface of the anode active material layers 34B to fabricate the anode 34. The charge and discharge conditions are as described above.

In a case where the aging treatment was performed on the secondary batteries, the secondary batteries were stored in a constant-temperature bath. The treatment temperature (° C.), the treatment time (time), the state of charge (%) of the secondary battery that are conditions of the aging treatment are as illustrated in Table 1. Thus, the laminated film type secondary batteries were completed.

It is to be noted that for comparison, a secondary battery was fabricated in a similar procedure, except that aging treatment was not performed. The presence or absence of the aging treatment is as illustrated in Table 1.

### [Fabrication of Coin Type Secondary Battery]

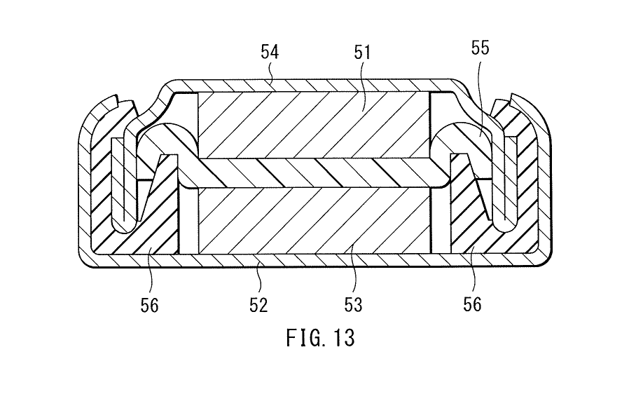

Moreover, a coin type secondary battery illustrated in FIG. 13 was fabricated as a test-use secondary battery.

In the secondary battery, a test electrode 51 was contained inside an outer package cup 54, and a counter electrode 53 was contained inside an outer package can 52. The test electrode 51 and the counter electrode 53 were stacked with a separator 55 in between, and the outer package can 52 and the outer package cup 54 were swaged with a gasket 56. Each of the test electrode 51, the counter electrode 53, and the separator 55 was impregnated with the electrolytic solution.

The secondary battery was fabricated as follows. The test electrode 51 was fabricated in a procedure similar to the procedure of fabricating the foregoing anode 34, except that the anode active material layer was formed only on a single surface of the anode current collector. As the counter electrode 53, lithium metal was used. The configuration of the separator 55 was similar to the configuration of the foregoing separator 35.

### [Measurement of Porosity]

After the fabrication of the laminated film type secondary batteries was completed, the secondary batteries was continuously charged, and then discharged in a procedure similar to a float test to be described later. The secondary batteries were continuously charged before measuring the porosity to accelerate breakdown and reformation of the coating film 34C, thereby setting strict porosity measurement conditions. In other words, in a case where the coating film 34C is broken down and reformed, the breakdown and reformation are easily repeated, which more easily causes the plurality of pores to be filled with the formation material of the coating film 34C. Thereafter, the anode 34 was collected from each of the secondary batteries.

Next, the anode 34 was immersed (for immersion time=one day) in an organic solvent (dimethyl carbonate) inside a glovebox (a total of an oxygen concentration and a water concentration≦100 ppm) to clean the anode 34. Subsequently, the anode 34 was taken out of the organic solvent, and thereafter, the anode 34 was dried (for drying time=one day) in a vacuum environment. Thereafter, a portion of the anode active material layer 34B was cut, and the porosity (%) of the portion of the anode active material layer 34B was measured. Thus, results illustrated in Table 1 were obtained. Details of the method of cutting the anode 34 and the method of measuring the porosity are as described above.

It is to be noted that in a case where the secondary batteries were fabricated, the foregoing aging treatment conditions (the treatment temperature, the treatment time, and the state of charge) were changed to change the porosity.

### [Analysis of Anode Using FT-IR]

After the fabrication of the secondary batteries was completed, in order to make the charged state uniform, the secondary batteries was charged and discharged, and then charged again in the following procedure.

First, three cycles of charge and discharge were performed on each of the secondary batteries in an ordinary temperature environment (at a temperature of 23° C.). In the first cycle and the second cycle of charge and discharge, the secondary battery was charged at a constant current of 0.1 C until the voltage reached 2.4 V, and thereafter, the secondary battery was charged at a constant voltage of 2.4 V until the current corresponded to 1/30 of an initial current (=0.1 C), and the secondary battery was discharged at a constant current of 0.1 C until the voltage reached 0.5 V. Conditions at the third cycle of charge and discharge were similar to conditions at the first cycle and the second cycle of charge and discharge, except that each of the current during charge and the current during discharge was changed to 0.2 C. It is to be noted that “0.2 C” refers to a current value at which the battery capacity is completely discharged in 5 hours. Subsequently, the secondary battery was charged and discharged in the same environment, and the discharge capacity of the secondary battery was measured. Charge and discharge conditions were similar to the charge and discharge conditions at the third cycle. Lastly, the secondary battery was charged in the same environment. In this case, in a case where the foregoing discharge capacity was considered as 100%, the secondary battery was charged at a constant current of 0.2 C until obtaining discharge capacity corresponding to 50% of the foregoing discharge capacity.

Thereafter, the anode 34 was collected from the secondary battery in the charged state, and the anode 34 (the coating film 34C) was analyzed with use of the FT-IR.

The presence or absence of a peak detected by surface analysis of the anode 34, that is, whether a peak was detected in each of the first range (<1000 cm⁻¹ ), the second range (>2000 cm⁻¹), and the third range (from 2000 cm⁻¹ to 1000 cm⁻¹ both inclusive) is as illustrated in Table 1. It is to be noted that details of the analysis apparatus and analysis conditions are as described above.

### [Evaluation of Secondary Batteries]

Cycle characteristics, electrical resistance characteristics, and swollenness characteristics were examined to evaluate battery characteristics of the secondary batteries, and results illustrated in Table 1 were thereby obtained.

The cycle characteristics were examined as follows. A cycle test was performed with use of the coin type secondary battery to determine a capacity retention ratio (%).

In the cycle test, first, one cycle of charge and discharge was performed on the secondary battery in an ordinary temperature environment (at a temperature of 23° C.) to measure discharge capacity (discharge capacity at the first cycle). When the secondary battery was charged, the secondary battery was charged at a constant current of 0.2 C until the voltage reached 2.4 V, and thereafter, the secondary battery was charged at a constant voltage of 2.4 V until the current corresponded to 1/30 of the initial current (=0.2 C). When the secondary battery was discharged, the secondary battery was discharged at a constant current of 0.2 V until the voltage reached 0.5 V.

Subsequently, the secondary battery was repeatedly charged and discharged until the total number of cycles reached 500 cycles in a high temperature environment (at a temperature of 45° C.). Charge and discharge conditions were similar to the charge and discharge conditions at the first cycle, except that each of the current during charge and the current during discharge was changed to 1 C. It is to be noted that “1 C” refers to a current value at which the battery capacity is completely discharged in 1 hour.

Subsequently, the secondary battery was charged and discharged in an ordinary temperature environment (at a temperature of 23° C.) to measure discharge capacity (discharge capacity at 501st cycle). Charge and discharge conditions were similar to the charge and discharge conditions at the first cycle.

Lastly, a capacity retention ratio (%)=(discharge capacity at the 501st cycle/discharge capacity at the first cycle)×100 was calculated.

Moreover, the electrical resistance characteristics were examined as follows. In a case where the coin type secondary battery was fabricated, electrochemical impedance (EIS (Ω)) of the test electrode 51 was measured with use of an alternating-current impedance method. The electrochemical impedance is so-called charge transfer resistance. As a measurement apparatus, a multi-channel potentiostat VMP-3 available from Bio-Logic Science Instruments SAS located in France was used. As measurement conditions, a frequency range was from 1 MHz to 10 MHz, an AC amplitude was 10 mV, and a DC voltage was 0V (OCV).

Further, the swollenness characteristics were examined as follows. The float test was performed with use of the laminated film type secondary battery to determine a volume change ratio (%).

In the float test, first, the secondary battery was charged and discharged at an ordinary temperature environment (at a temperature of 23° C.) to measure discharge capacity. Charge and discharge conditions were similar to the charge and discharge conditions (at the first cycle) in a case where the cycle characteristics were examined.

Subsequently, the secondary battery was charged again, and thereafter, a volume of the secondary battery in such a charged state (a volume before continuous charge) was measured. In this case, in a case where the foregoing discharge capacity was considered as 100%, the secondary battery was charged at a constant current of 0.2 C until obtaining discharge capacity corresponding to 50% of the foregoing discharge capacity.

It is to be noted that a procedure of measuring the volume of the secondary battery is as described below. First, a beaker containing water was put on an electronic balance. In this case, capacity of water was about 80% of capacity of the beaker. Subsequently, the secondary battery was completely immersed in water contained in the beaker. Lastly, the volume of the secondary battery was determined on the basis of an increase in weight after immersion of the secondary battery. This procedure of measuring the volume was similarly used in the following procedure.

Thereafter, the secondary battery continued being charged at an ordinary temperature environment (at a temperature of 23° C.) to measure discharge capacity. In this case, the secondary battery was charged at a constant current of 0.2 C until the voltage reached 2.4 V. In other words, as described above, the secondary battery was charged at a constant current until obtaining discharge capacity corresponding to 50%, and thereafter, the secondary battery continued being charged at a constant current until obtaining discharge capacity corresponding to 100%.

Subsequently, the secondary battery was continuously charged at a high temperature environment (at a temperature of 45° C.). In this case, the secondary battery was charged at a constant voltage of 2.4 V until charge time reached 500 hours. Thereafter, the secondary battery was discharged in an ordinary temperature environment (at a temperature of 23° C.). In this case, the secondary battery was discharged at a constant current of 0.2 C until the voltage reached 0.5 V.

Subsequently, the secondary battery was charged and discharged in the same environment. The charge and discharge conditions were similar to the charge and discharge conditions (at the first cycle) in the case where the cycle characteristics were determined.

Subsequently, the secondary batteries were charged again, and the volume of the secondary battery in such a charged state (volume after continuous charge) was measured. In this case, in a case where the foregoing discharge capacity was considered as 100%, the secondary battery was charged at a constant current of 0.2 C until obtaining discharge capacity corresponding to 50% of the foregoing discharge capacity.

Lastly, a volume change ratio (%)=[(the volume after continuous charge□ the volume before continuous charge)/the volume before continuous charge]×100 was calculated.

TABLE 1
Anode Active Material: Lithium-Titanium Composite Oxide (Li₄Ti₅O₁₂)

| | Unsaturated | | | | | | | | | | | | |
|---|---|---|---|---|---|---|---|---|---|---|---|---|---|
| | Cyclic | | | | | | | | | | | | |
| | Carbonate | | | | | | | Peak | | | Capacity | | Volume |
| | Ester | | Aging Treatment | | | | | (FT-IR) | | | Retention | | Change |
| Experimental | | Content | Presence or | Temperature | Time | SOC | Porosity | First | Second | Third | Ratio | EIS | ratio |
| Example | Kind | (wt %) | Absence | (° C.) | (Hour) | (%) | (%) | Range | Range | Range | (%) | (Ω) | (%) |
| 1-1 | — | — | Absence | — | — | — | 23 | Detected | Detected | Detected | 58 | 75 | 96 |
| 1-2 | — | — | Presence | 45 | 48 | 50 | 18 | Detected | Detected | Detected | 56 | 77 | 112 |
| 1-3 | VC | 1 | Absence | — | — | — | 24 | Detected | Detected | Detected | 58 | 61 | 86 |
| 1-4 | VC | 1 | Presence | 25 | 48 | 50 | 23 | Detected | Detected | Detected | 58 | 62 | 90 |
| 1-5 | VC | 1 | Presence | 45 | 48 | 10 | 24 | Detected | Detected | Detected | 58 | 65 | 92 |
| 1-6 | VC | 1 | Presence | 45 | 48 | 25 | 43 | Detected | Detected | Not | 64 | 43 | 67 |
| | | | | | | | | | | Detected | | | |
| 1-7 | VC | 0.01 | Presence | 45 | 48 | 50 | 36 | Detected | Detected | Not | 60 | 55 | 85 |
| | | | | | | | | | | Detected | | | |
| 1-8 | VC | 1 | Presence | 45 | 48 | 50 | 50 | Detected | Detected | Not | 65 | 48 | 67 |
| | | | | | | | | | | Detected | | | |
| 1-9 | VC | 2 | Presence | 45 | 48 | 50 | 41 | Detected | Detected | Not | 63 | 45 | 59 |
| | | | | | | | | | | Detected | | | |
| 1-10 | VC | 5 | Presence | 45 | 48 | 50 | 30 | Detected | Detected | Not | 60 | 57 | 69 |
| | | | | | | | | | | Detected | | | |
| 1-11 | VC | 1 | Presence | 45 | 48 | 75 | 42 | Detected | Detected | Not | 64 | 47 | 65 |
| | | | | | | | | | | Detected | | | |
| 1-12 | VC | 1 | Presence | 45 | 12 | 50 | 41 | Detected | Detected | Not | 62 | 46 | 69 |
| | | | | | | | | | | Detected | | | |
| 1-13 | VC | 1 | Presence | 45 | 100 | 50 | 42 | Detected | Detected | Not | 61 | 46 | 70 |
| | | | | | | | | | | Detected | | | |
| 1-14 | VC | 1 | Presence | 45 | 48 | 90 | 24 | Detected | Detected | Detected | 57 | 62 | 87 |
| 1-15 | VC | 1 | Presence | 60 | 48 | 50 | 40 | Detected | Detected | Detected | 62 | 49 | 69 |
| 1-16 | VC | 1 | Presence | 80 | 48 | 50 | 23 | Detected | Detected | Detected | 55 | 71 | 87 |

### [Consideration]

As illustrated in Table 1, in the case where the titanium-containing compound (the lithium-titanium composite oxide) was used as the anode active material, a relationship between the porosity and all of the capacity retention ratio, the EIS, and the volume change ratio largely varied depending on the presence or absence of the aging treatment and conditions of the aging treatment.

More specifically, in a case where the electrolytic solution did not include the unsaturated cyclic carbonate ester (experimental examples 1-1 and 1-2), independently of presence or absence of the aging treatment, the porosity was decreased and a peak was detected in each of the first range, the second range, and the third range. In this case, in a case where the aging treatment was performed (the experimental example 1-2), as compared with a case where the aging treatment was not performed (the experimental example 1-1), the capacity retention ratio was decreased, and each of the EIS and the volume change ratio was increased.

In contrast, in a case where the electrolytic solution included the unsaturated cyclic carbonate ester (experimental examples 1-3 to 1-16), each of the capacity retention ratio, the EIS, and the volume change ratio was improved depending on presence or absence of the aging treatment and conditions of the aging treatment.

More specifically, in a case where the electrolytic solution included the unsaturated cyclic carbonate ester but the conditions of the aging treatment were not appropriate (experimental example 1-4, 1-5, 1-14, and 1-16), as with a case where the aging treatment was not performed (the experimental example 1-3), the porosity was still low, and a peak was detected in each of the first range, the second range, and the third range. In this case, in a case where the aging treatment was performed, as compared with the case where the aging treatment was not performed, the capacity retention ratio was substantially equal or smaller, and each of the EIS and the volume change ratio was increased.

However, in a case where the electrolytic solution included the unsaturated cyclic carbonate ester and the aging treatment conditions were appropriate (the experimental examples 1-6 to 1-13 and 1-15), the porosity was increased, and while a peak was detected in each of the first range and the second range, a peak was not detected in the third range. In this case, in the case where the aging treatment was performed, as compared with the case where the aging treatment was not performed, the capacity retention ratio was increased, and each of the EIS and the volume change ratio was decreased.

As the appropriate conditions of the aging treatment, the treatment temperature was within a range from 45° C. to 60° C. both inclusive, the treatment time was within a range from 12 hours to 100 hours both inclusive, and the state of charge of the secondary battery was within a range from 25% to 75% both inclusive. Moreover, the porosity in the case where the aging treatment was performed under appropriate conditions was within a range from 30% to 50% both inclusive.

In the case where the electrolytic solution included the unsaturated cyclic carbonate ester, the following tendency was derived from these results. Even if the aging treatment was performed, in a case where the conditions of the aging treatment were not appropriate, the porosity was still low, which deteriorated the capacity retention ratio, the EIS, and the volume change ratio.

In contrast, in the case where the aging treatment was performed under the appropriate conditions, the porosity was increased, which improved the capacity retention ratio, the EIS, and the volume change ratio.

Accordingly, it was considered that performing the aging treatment under the appropriate conditions made the state (physical properties) of the coating film 34C appropriate, thereby easily keeping the plurality of pores unfilled, which suppressed a decrease in the capacity retention ratio, and suppressed an increase in each of the EIS and the volume change ratio.

### (Experimental Examples 2-1 to 2-4)

Secondary batteries were fabricated in a similar procedure, except that a carbon material (graphite) was used as the anode active material in place of the titanium-containing compound as illustrated in Table 2, and thereafter, battery characteristics of the secondary batteries were examined. In this case, volume density of the cathode active material layer 33B was 1.8 g/cm³, and volume density of the anode active material layer 34B was 1.4 g/cm³.

TABLE 2
Anode Active Material: Carbon Material (Graphite)

| | Unsaturated | | | | | | | | | | | | |
|---|---|---|---|---|---|---|---|---|---|---|---|---|---|
| | Cyclic | | | | | | | | | | | | |
| | Carbonate | | | | | | | Peak | | | Capacity | | Volume |
| | Ester | | Aging Treatment | | | | | (FT-IR) | | | Retention | | Change |
| Experimental | | Content | Presence or | Temperature | Time | SOC | Porosity | First | Second | Third | Ratio | EIS | ratio |
| Example | Kind | (wt %) | Absence | (° C.) | (Hour) | (%) | (%) | Range | Range | Range | (%) | (Ω) | (%) |
| 2-1 | — | — | Absence | — | — | — | 26 | Detected | Detected | Detected | 44 | 72 | 70 |
| 2-2 | — | — | Presence | 45 | 48 | 50 | 25 | Detected | Detected | Detected | 41 | 72 | 75 |
| 2-3 | VC | 1 | Absence | — | — | — | 29 | Detected | Detected | Detected | 48 | 69 | 69 |
| 2-4 | VC | 1 | Presence | 45 | 48 | 50 | 27 | Detected | Detected | Detected | 48 | 69 | 69 |

As illustrated in Table 2, in a case where the carbon material was used as the anode active material (experimental examples 2-1 to 2-4), independently of the presence or absence of the unsaturated cyclic carbonate ester and the presence or absence of the aging treatment, the porosity was decreased, and a peak was detected in each of the first range, the second range, and the third range. In this case, in a case where the aging treatment was performed (the experimental examples 2-2 and 2-4), as compared with a case where the aging treatment was not performed (the experimental example 2-1 and 2-3), the capacity retention ratio was substantially equal or smaller, EIS was substantially equal, and the volume change ratio were substantially equal or larger.

From this result, an advantageous tendency that in a case where the electrolytic solution includes the unsaturated cyclic carbonate ester and the aging treatment is performed under appropriate conditions, initial pores (upon formation of the anode active material layer 34B) are maintained easily, thereby obtaining favorable results of the capacity retention ratio, the EIS, and the volume change ratio is considered as a specific tendency obtained only in a case where the titanium-containing compound is used as the anode active material.

As can be seen from the results illustrated in Tables 1 and 2, in the case where the anode included the titanium-containing compound, the electrolytic solution included the unsaturated cyclic carbonate ester, and the foregoing porosity of the portion of the anode active material layer was within a range from 30% to 50% both inclusive, all of the cyclic characteristics, the electrical resistance characteristics, and the swollenness characteristics were improved. Accordingly, superior battery characteristics were obtained in the secondary battery.

Although the present technology has been described above referring to some embodiments and examples, the present technology is not limited thereto, and may be modified in a variety of ways.

More specifically, the description has been given with reference to the cylindrical type secondary battery, the laminated film type secondary battery, and the coin type secondary battery as examples of the secondary battery of the present technology. However, the secondary battery of the present technology may be any other secondary battery. Non-limiting examples of the other secondary battery may include a square type secondary battery.

Moreover, description has been given with reference to an example in which the battery element has the spirally wound structure. However, the structure of the battery element in the secondary battery of the present technology is not particularly limited. More specifically, the battery element may have any other structure such as a stacked structure.

Note that the effects described in the present specification are illustrative and non-limiting. The technology may have effects other than those described in the present specification.

It is to be noted that the present technology may have the following configurations.
(1)

A secondary battery, including:

a cathode;

an anode including an anode active material layer and a coating film, the anode active material layer including a titanium-containing compound, and a surface of the anode active material layer being coated with the coating film; and

an electrolytic solution including one or more of respective unsaturated cyclic carbonate esters represented by the following formulas (11) to (13),

in which porosity of a portion of the anode active material layer measured with use of a mercury intrusion technique is within a range from 30% to 50% both inclusive, and the portion of the anode active material layer is cut together with a portion of the coating film from a surface of the coating film to a depth of 10 μm,

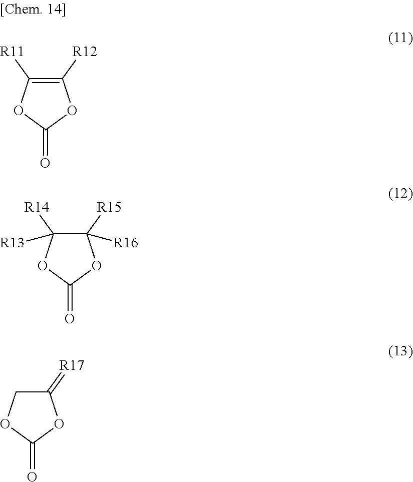

where each of R11 and R12 is one of a hydrogen group and an alkyl group, each of R13 to R16 is one of a hydrogen group, an alkyl group, a vinyl group, and an allyl group, one or more of R13 to R16 are one of the vinyl group and the allyl group, R17 is a group represented by >CR171R172, and each of R171 and R172 is one of a hydrogen group and an alkyl group.
(2)

The secondary battery according to (1), in which a peak is detected by analysis of the coating film with use of Fourier transform infrared spectroscopy in each of a wave number range smaller than 1000 cm⁻¹ , and a wave number range larger than 2000 cm⁻¹ , and a peak is not detected in a wave number range from 1000 cm⁻¹ to 2000 cm⁻¹ both inclusive.
(3)

The secondary battery according to (1) or (2), in which the titanium-containing compound includes one or more of a titanium oxide represented by the following formula (1) and respective lithium-titanium composite oxides represented by the following formulas (2) to (4),

TiO_{w} (1)

where w satisfies 1.85≦w≦2.15.

Li[LiₓM1_{(1-3x)/2}Ti_{(3+x)/2}]O₄ (2)

where M1 is one or more of magnesium (Mg), calcium (Ca), copper (Cu), zinc (Zn), and strontium (Sr), and “x” satisfies 0≦x≦1/3,

Li[Li_{y}M2_{1-3y}Ti_{1+2y}]O₄ (3)

where M2 is one or more of aluminum (Al), scandium (Sc), chromium (Cr), manganese (Mn), iron (Fe), germanium (Ga), and yttrium (Y), and “y” satisfies 0≦y≦1/3, and

Li[Li_{1/3}M3_{z}Ti_{(5/3)-z}]O₄ (4)

where M3 is one or more of vanadium (V), zirconium (Zr), and niobium (Nb), and “z” satisfies 0≦z2/3.
(4)

The secondary battery according to any one of (1) to (3), in which the unsaturated cyclic carbonate esters include vinylene carbonate.
(5)

The secondary battery according to any one of (1) to (4), in which a content of the unsaturated cyclic carbonate esters in the electrolytic solution is within a range from 0.01 wt % to 5 wt % both inclusive.
(6)

The secondary battery according to any one of (1) to (5), in which a thickness of the coating film is 100 nm or less.
(7)

The secondary battery according to any one of (1) to (6), in which a capacity retention ratio after 500 cycles of charge and discharge are performed in an environment at 45° C. is 60% or more.
(8)

The secondary battery according to any one of (1) to (7), in which electrochemical impedance of the anode measured with use of an alternate-current impedance method is 57 Ω or less.
(9)

The secondary battery according to any one of (1) to (8), in which a volume change ratio after the secondary battery is continuously charged in an environment at 45° C. until charge time reaches 500 hours is 85% or less.
(10)

The secondary battery according to any one of (1) to (9), in which the secondary battery is a lithium-ion secondary battery.
(11)

A method of manufacturing a secondary battery, comprising:

fabricating a secondary battery including a cathode, an anode, and an electrolytic solution, the anode including an anode active material layer that includes a titanium-containing compound, and the electrolytic solution including one or more of respective unsaturated cyclic carbonate esters represented by the following formulas (11) to (13);

charging and discharging the secondary battery to form a coating film, a surface of the anode active material layer being coated with the coating film; and

performing heat treatment on the secondary battery, in which the coating film is formed on the surface of the anode active material layer, at a treatment temperature ranging from 45° C. to 60° C. both inclusive for treatment time ranging from 12 hours to 100 hours both inclusive in a state of charge ranging from 25% to 75% both inclusive,

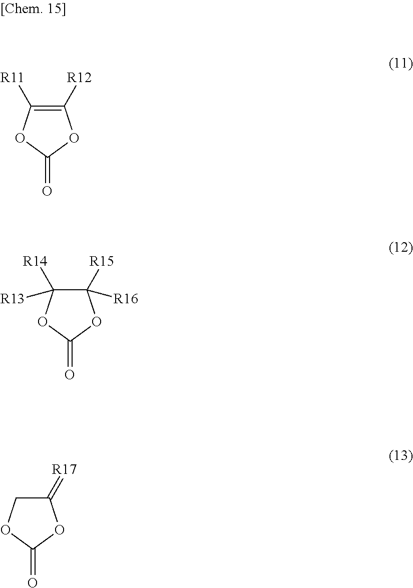

where each of R11 and R12 is one of a hydrogen group and an alkyl group, each of R13 to R16 is one of a hydrogen group, an alkyl group, a vinyl group, and an allyl group, one or more of R13 to R16 are one of the vinyl group and the allyl group, R17 is a group represented by >CR171R172, and each of R171 and R172 is one of a hydrogen group and an alkyl group.
(12)

A battery pack, including:

the secondary battery according to any one of (1) to (10);

a controller that controls an operation of the secondary battery; and

a switch section that switches the operation of the secondary battery in accordance with an instruction from the controller.
(13)

An electric vehicle, including:

the secondary battery according to any one of (1) to (10);

a converter that converts electric power supplied from the secondary battery into drive power;

a drive section that operates in accordance with the drive power; and

a controller that controls an operation of the secondary battery.
(14)

An electric power storage system, including:

the secondary battery according to any one of (1) to (10);

one or more electric devices that are supplied with electric power from the secondary battery; and

a controller that controls the supplying of the electric power from the secondary battery to the one or more electric devices.
(15)

An electric power tool, including:

the secondary battery according to any one of (1) to (10); and

a movable section that is supplied with electric power from the secondary battery.
(16)

An electronic apparatus including the secondary battery according to any one of (1) to (6) as an electric power supply source.

It should be understood by those skilled in the art that various modifications, combinations, sub-combinations and alterations may occur depending on design requirements and other factors insofar as they are within the scope of the appended claims or the equivalents thereof.

What is claimed is:

1. A secondary battery, comprising:
a cathode;
an anode including an anode active material layer and a coating film, the anode active material layer including a titanium-containing compound, and a surface of the anode active material layer being coated with the coating film; and
an electrolytic solution including one or more of respective unsaturated cyclic carbonate esters represented by the following formulas (11) to (13),
wherein porosity of a portion of the anode active material layer measured with use of a mercury intrusion technique is within a range from 30% to 50% both inclusive, and the portion of the anode active material layer is cut together with a portion of the coating film from a surface of the coating film to a depth of 10 μm, 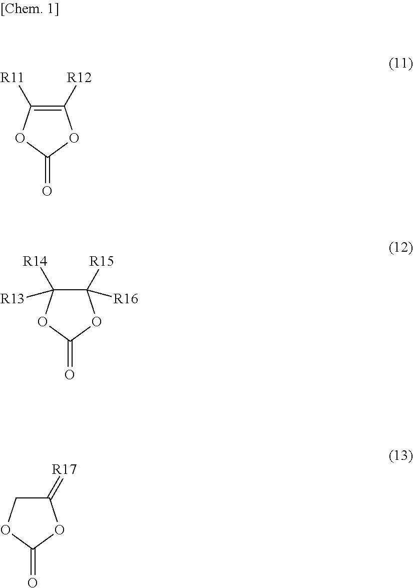
where each of R11 and R12 is one of a hydrogen group and an alkyl group, each of R13 to R16 is one of a hydrogen group, an alkyl group, a vinyl group, and an allyl group, one or more of R13 to R16 are one of the vinyl group and the allyl group, R17 is a group represented by >CR171R172, and each of R171 and R172 is one of a hydrogen group and an alkyl group.

2. The secondary battery according to claim 1, wherein a peak is detected by analysis of the coating film with use of Fourier transform infrared spectroscopy in each of a wave number range smaller than 1000 cm⁻¹, and a wave number range larger than 2000 cm⁻¹, and a peak is not detected in a wave number range from 1000 cm⁻¹ to 2000 cm⁻¹ both inclusive.

3. The secondary battery according to claim 1, wherein the titanium-containing compound includes one or more of a titanium oxide represented by the following formula (1) and respective lithium-titanium composite oxides represented by the following formulas (2) to (4),
TiO_{w} (1)
where w satisfies 1.85≦w2.15.
Li[LiₓM1_{(1-3x)/2}Ti_{(3+x)/2}]O₄ (2)
where M1 is one or more of magnesium (Mg), calcium (Ca), copper (Cu), zinc (Zn), and strontium (Sr), and “x” satisfies 0≦x≦1/3,
Li[Li_{y}M2_{1-3y}Ti₁₊₂₃] O₄ (3)
where M2 is one or more of aluminum (Al), scandium (Sc), chromium (Cr), manganese (Mn), iron (Fe), germanium (Ga), and yttrium (Y), and “y” satisfies 0≦y≦1/3, and
Li[Li_{1/3}M3_{z}Ti_{(5/3)-z}]O₄ (4)
where M3 is one or more of vanadium (V), zirconium (Zr), and niobium (Nb), and “z” satisfies 0≦z≦2/3.

4. The secondary battery according to claim 1, wherein the unsaturated cyclic carbonate esters include vinylene carbonate.

5. The secondary battery according to claim 1, wherein a content of the unsaturated cyclic carbonate esters in the electrolytic solution is within a range from 0.01 wt % to 5 wt % both inclusive.

6. The secondary battery according to claim 1, wherein a thickness of the coating film is 100 nm or less.

7. The secondary battery according to claim 1, wherein a capacity retention ratio after 500 cycles of charge and discharge are performed in an environment at 45° C. is 60% or more.

8. The secondary battery according to claim 1, wherein electrochemical impedance of the anode measured with use of an alternate-current impedance method is 57 Ω or less.

9. The secondary battery according to claim 1, wherein a volume change ratio after the secondary battery is continuously charged in an environment at 45° C. until charge time reaches 500 hours is 85% or less.

10. The secondary battery according to claim 1, wherein the secondary battery is a lithium-ion secondary battery.

11. A method of manufacturing a secondary battery, comprising:
fabricating a secondary battery including a cathode, an anode, and an electrolytic solution, the anode including an anode active material layer that includes a titanium-containing compound, and the electrolytic solution including one or more of respective unsaturated cyclic carbonate esters represented by the following formulas (11) to (13);
charging and discharging the secondary battery to form a coating film, a surface of the anode active material layer being coated with the coating film; and
performing heat treatment on the secondary battery, in which the coating film is formed on the surface of the anode active material layer, at a treatment temperature ranging from 45° C. to 60° C. both inclusive for treatment time ranging from 12 hours to 100 hours both inclusive in a state of charge ranging from 25% to 75% both inclusive, 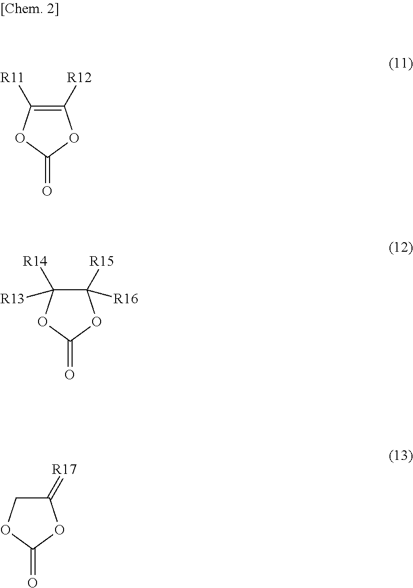
where each of R11 and R12 is one of a hydrogen group and an alkyl group, each of R13 to R16 is one of a hydrogen group, an alkyl group, a vinyl group, and an allyl group, one or more of R13 to R16 are one of the vinyl group and the allyl group, R17 is a group represented by >CR171R172, and each of R171 and R172 is one of a hydrogen group and an alkyl group.

12. An electronic apparatus comprising a secondary battery as an electric power supply source, the secondary battery including
a cathode,
an anode including an anode active material layer and a coating film, the anode active material layer including a titanium-containing compound, and a surface of the anode active material layer being coated with the coating film, and
an electrolytic solution including one or more of respective unsaturated cyclic carbonate esters represented by the following formulas (11) to (13),
wherein porosity of a portion of the anode active material layer measured with use of a mercury intrusion technique is within a range from 30% to 50% both inclusive, and the portion of the anode active material layer is cut together with a portion of the coating film from a surface of the coating film to a depth of 10 μm, 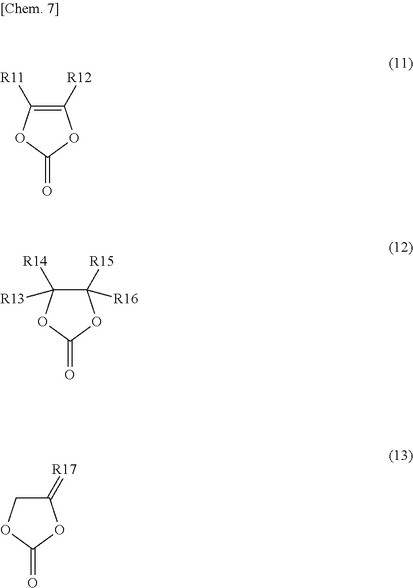
where each of R11 and R12 is one of a hydrogen group and an alkyl group, each of R13 to R16 is one of a hydrogen group, an alkyl group, a vinyl group, and an allyl group, one or more of R13 to R16 are one of the vinyl group and the allyl group, R17 is a group represented by >CR171R172, and each of R171 and R172 is one of a hydrogen group and an alkyl group.
